(19) United States
(12) Patent Application Publication
(10) Pub. No.: US 2023/0294064 A1
(43) Pub. Date: Sep. 21, 2023

(54) SUBSTRATES, SYSTEMS, AND METHODS FOR NUCLEIC ACID ARRAY SYNTHESIS

(71) Applicant: VIBRANT HOLDINGS, LLC, San Carlos, CA (US)

(72) Inventor: John J. Rajasekaran, Hillsborough, CA (US)

(21) Appl. No.: 18/106,443

(22) Filed: Feb. 6, 2023

Related U.S. Application Data
(63) Continuation of application No. 16/060,214, filed on Jun. 7, 2018, now abandoned.
(60) Provisional application No. 62/272,057, filed on Dec. 28, 2015.

(51) Int. Cl. B01J 19/00; C12Q 1/6837; C40B 50/14; G03F 7/004; G03F 7/20; G03F 7/42; C40B 50/00; C12Q 1/68

(52) U.S. Cl. CPC B01J 19/0046; C12Q 1/6837; C40B 50/14; G03F 7/0045; G03F 7/2004; G03F 7/42; C40B 50/00; C12Q 1/68; B01J 2219/00432; B01J 2219/00504; B01J 2219/00529; B01J 2219/00533; B01J 2219/00596; B01J 2219/00626; B01J 2219/00659; B01J 2219/00675; B01J 2219/00711; B01J 2219/00729; G03F 7/162

(57) ABSTRACT

Disclosed herein are formulations, substrates, and arrays for the synthesis of PNA chains and PNA-DNA chimera on microarrays. In some embodiments, the formulations include a photo-protective compound that shields any PNA monomers, PNA polymers, or PNA-DNA chimera already attached to a microarray from radiation exposure during the synthesis of the PNA or PNA-DNA chains. In some embodiments, substrates and arrays comprise a porous or a planar layer for synthesis and attachment of PNA or DNA monomers, or PNA or PNA-DNA polymers. In some embodiments, disclosed herein are formulations and methods for high efficiency coupling of PNA monomers or PNA polymers to a microarray substrate.

### CROSS-REFERENCE TO RELATED APPLICATIONS

This application is a continuation of U.S. national phase application Ser. No. 16/060,214, filed Jun. 7, 2018, which claims priority to PCT/US2016/69017, filed on Dec. 28, 2016, which claims the benefit of U.S. Provisional Patent Application No. 62/272,057, filed Dec. 28, 2015, the disclosure of which is incorporated herein by reference.

### BACKGROUND

A typical microarray system is generally comprised of biomolecular probes, such as DNA or RNA, formatted on a solid planar surface like glass, plastic, or silicon chip, plus the instruments needed to handle samples (automated robotics), to read the reporter molecules (scanners) and analyze the data (bioinformatic tools). Microarray technology can facilitate monitoring of many probes per square centimeter. Advantages of using multiple probes include, but are not limited to, speed, adaptability, comprehensiveness and the relatively cheaper cost of high volume manufacturing. The uses of such an array include, but are not limited to, diagnostic microbiology, including the detection and identification of pathogens, investigation of anti-microbial resistance, epidemiological strain typing, investigation of oncogenes, analysis of microbial infections using host genomic expression, and polymorphism profiles.

Peptide Nucleic Acids (PNAs) are DNA analogues with a peptide-like backbone with each subunit containing a naturally occurring or non-naturally occurring base. One such backbone is constructed of repeating units of N-(2-aminoethyl) glycine linked through amide bond as described in P. E. Nielsen et al., Science, 254, 1497-1500 (1991). U.S. Pat. No. 6,395,474 further discloses with respect to the new class of compounds (PNA), which binds both DNA and RNA, the formation of PNA/DNA or PNA/RNA duplexes. PNA/oligonucleotide hybrids are thermally more stable than the corresponding DNA (or RNA) hybrids, and they possess increased biological stability. The specificity of binding towards DNA and RNA has opened the way to biotechnological applications of PNAs, including the identification of single nucleotide polymorphisms in PCR-based assays. PNA has the potential to be a highly sensitive method for direct detection of the binding to RNA or DNA. However, for a robust analysis, relatively large numbers of high quality oligomers arranged in a microarray format can significantly reduce the reaction volume and simplify the detection process.

Two methods for synthesizing PNA microarrays are well known to one skilled in the art, which include: (1) synthesizing different single probes, respectively, based on solid-phase synthesis technology, and then binding those probes at different locations on the microarray substrate by spot synthesis or by adsorption; and (2) employing UV light-directed photolithographic synthesis in situ using photomasks.

Examples of the first method are disclosed in U.S. Patent Publ. No. 2006/0147949, including the production of a PNA chip in which a probe PNA containing a desired DNA sequence is immobilized on a plastic substrate coated with an epoxy group-containing polymer by means of an epoxy group-containing polymer layer in an efficient and cost-effective manner. However, this method has shortcomings that include, but are not limited to, being a time-consuming process with low spatial resolution and high cost for synthesis of the single probes to form the PNA array.

Examples of the second method are disclosed in U.S. Pat. No. 6,359,125, which includes a process for preparing arrays of PNA probes immobilized on a solid matrix by employing polymeric photoacid generator. The speed of this process is increased as compared to the first method by using parallel processing when synthesizing the probes on the microarray. However, a limiting factor of this process entails the high synthesis cost of PNA monomers required for parallel synthesis.

Thus, both methods in the prior suffer from various deficiencies, including low or inconsistent coupling efficiencies across multiple coupling cycles, cost of synthesis, and difficulty to spatially control the synthesis of different PNA probes among others. In addition, photolithographic methods suffer from the effect that nucleic acids are instable (i.e., destroyed) when exposed to short wavelength radiation (e.g., UV radiation at 248 nm), which is required to obtain a higher PNA probe density on the microarray.

PNA-DNA chimera are oligomer molecules with distinct PNA and nucleotide moieties. They can be synthesized by covalently linking a sequence of PNA monomers along with a sequence of nucleotides in virtually any combination or sequence.

Egholm et al. (U.S. Pat. No. 6,316,230) provides methods and a kit for primer extension of PNA-DNA chimera from template nucleic acids using polymerases, nucleotide 5′-triphosphates, and primer extension reagents. The invention is based on the discovery that a PNA-DNA chimera can conduct primer extension under a broad range of experimental conditions and variables. DNA sequencing methods may benefit from the use of PNA-DNA chimera, where the increased affinity and specificity conferred by the PNA moiety in a PNA-DNA chimera allows for greater specificity. This method has shortcomings in that it has a low spatial resolution and high cost for synthesis of the single probes to form the PNA-DNA chimera array apart from being a time-consuming process.

What is needed, therefore, are methods and compositions that address these and other shortcomings for both PNA and PNA-DNA chimera probe synthesis on a microarray, yet still allow the arrays to be successfully used, e.g., for SNP detection from a sample.

### SUMMARY

Provided herein are methods and compositions that improve PNA and PNA-DNA chimera synthesis on an array and methods of use of these arrays. The present invention provides novel substrates, systems, and methods for nucleic acid microarray synthesis as described in detail below. In particular, the present invention includes a novel method of synthesizing sub monomer derived UV light directed PNA monomers and polymers on addressable locations of the microarray that is capable of resulting economic preparation of PNA microarrays with a high spot density of PNA probes.

In some embodiments, disclosed herein are arrays and methods of manufacturing arrays with high spot density and economic preparation of PNA and PNA-DNA chimeric probes. Here, we present a novel method of synthesizing sub monomer derived UV light directed PNA monomers and polymers followed by UV light directed DNA oligomers and polymers on addressable locations. This will combine the advantages of PNA (high specificity and coupling yields) along with the advantages of DNA oligomer (which allows detection using a PCR reaction on chip) thereby enabling better accuracy of detection.

Embodiments of the invention include formulations, substrates, and arrays. Embodiments also include methods for manufacturing and using the formulations, substrates, and arrays.

In some embodiments, provided, herein is an array of features attached to a surface at positionally-defined locations, each of said features comprising a plurality of PNA polymers of determinable sequence and intended length, wherein said plurality of PNA polymers comprises a distribution of lengths characterized by a coupling efficiency of at least 90%, 91%, 92%, 93%, 94%, 95%, 96%, 97%, 98%, 98.5%, 99%, or 99.5%. In some embodiments, the distribution of lengths of said plurality of PNA polymers less than the intended length is characterized by the equation F(N)=10^{(N+1)·log (E/100%)}−10^{(N)·log (E/100%)}, wherein N=the actual length of the PNA polymer and E=coupling efficiency, wherein E is at least 90%, 91%, 92%, 93%, 94%, 95%, 96%, 97%, 98%, 98.5%, 99%, or 99.5%. In some embodiments, the proportion of PNA polymers of intended length is characterized by the equation: F(N)=10^{(N)·log (E/100%)}, wherein N=the intended length of the PNA polymer and E=average coupling efficiency, wherein E is at least 90%, 91%, 92%, 93%, 94%, 95%, 96%, 97%, 98%, 98.5%, 99%, or 99.5%.

In some embodiments, the array comprises at least 10,000 features. In some embodiments, the array comprises at least 100,000 features.

In some embodiments, the intended length of the PNA polymer on the array is at least 30. In some embodiments, the intended length of the PNA polymer on the array is at least 40. In some embodiments, the intended length of the PNA polymer on the array is at least 50. In some embodiments, the intended length of the PNA polymer on the array is at least 75.

In some embodiments, the PNA polymers on the array are PNA/Nucleic Acid chimeras, wherein the PNA polymers further comprise one or more nucleic acid residues. In some embodiments, the nucleic acid is deoxyribonucleic acid.

In some embodiments, the array comprises at least 10,000 features per square centimeter. In some embodiments, the array comprises at least 20,000, at least 40,000, at least 100,000, at least 200,000, at least 500,000, at least 1 million, at least 2 million, at least 5 million, at least 10 million, at least 20 million, or at least 50 million features per square centimeter. In some embodiments, the array comprises pillars and said surface is the top surface of said pillars. In some embodiments, the top surface of the pillar has an area of at least 1 μm².

Also provided herein is a method of making a PNA or PNA-DNA chimera array, comprising generating a pattern with a photomask, exposing a photoresist to UV light through said photomask and generating an acid or a base in said pattern on said array as a result of said exposure to said UV light.

In some embodiments, the UV exposure generates a base from a photobase generator. In some embodiments, the photobase generator is selected from the group consisting of: 1,3-Bis[(2-nitrobenzyl)oxycarbonyl-4-piperidyl]propane, 1,3-Bis[1-(9-fluorenylmethoxycarbonyl)-4-piperidyl]propane, 1,5,7- triazabicyclo[4.4.0]dec-5-enyl-phenylglyoxylate, 1,5,7-triazabicyclo[4.4.0]dec-5- enyl-4- nitrophenylglyoxylate, 1,5,7-triazabicyclo[4.4.0]dec-5-enyl-tetraphenylborate, 1,8-Diazabicyclo[5.4.0]undec-7-enyl-tetraphenylborate, 1-Phenacyl-(1-azonia-4- azabicyclo[2,2,2]octane)-tetraphenylborate, and 1-Naphthoylmethyl-(1- azonia-4-azabicyclo[2,2,2]octane)-tetraphenylborate or similar. In some embodiments, the photobase generator is 1,3-Bis[(2-nitrobenzyl)oxycarbonyl-4-piperidyl]propane. In some embodiments, the base from the photobase generator cleaves a protecting group from an amine group.

In some embodiments, the method of making a PNA or PNA-DNA chimera array comprises coupling a PNA monomer to the deprotected amine group.

In some embodiments, the UV exposure generates an acid from a photoacid generator. In some embodiments, the photoacid generator is selected from the group consisting of an iodonium salt, a polonium salt, and a sulfonium salt. In some embodiments, the photoacid generator is a Bis(4-tert-butylphenyl)iodonium perfluoro-1-butanesulfonate, Bis(4-tert-butylphenyl)iodonium p-toluenesulfonate, Bis(4-tert-butylphenyl)iodonium triflate, Boc-methoxyphenyldiphenylsulfonium triflate, (tert-Butoxycarbonylmethoxynaphthyl)-diphenylsulfonium triflate, (4-tert-Butylphenyl)diphenylsulfonium triflate, Diphenyliodonium hexafluorophosphate, Diphenyliodonium perfluoro-1-butanesulfonate, Diphenyliodonium triflate, (4-Iodophenyl)diphenylsulfonium triflate, (4-Methoxyphenyl)diphenylsulfonium triflate, (4-Methylphenyl)diphenylsulfonium triflate, (4-Methylthiophenyl)methyl phenyl sulfonium triflate, Tris(4-tert-butylphenyl)sulfonium triflate, (4-Methoxyphenyl)phenylsulfonium triflate, (4-Methoxyphenyl)phenyliodonium triflate, 4 Methoxyphenyl)phenyliodonium trifluoromethanesulfonate, (4 methoxyphenyl)dimethylsulfonium triflate, (2,4-dihydroxyphenyl)dimethylsulfonium triflate or similar. In some embodiments, the photoacid generator is an iodonium or sulfonium salt of triflate, phosphate or antimonates. In some embodiments, the photoacid generator is (4-Iodophenyl)diphenylsulfonium triflate. In some embodiments, the acid generated by the photoacid generator cleaves a protecting group from a carboxylic acid group.

In some embodiments, the method of creating a PNA-DNA chimera array further comprises coupling a DNA monomer to the deprotected carboxylic acid group. In some embodiments, coupling of the PNA monomer comprises activating a substituted acetic acid by an activation agent, and coupling of the activated acetic acid to the unprotected amine groups at said selectively exposed area, wherein the substitution of the acetic acid comprises a leaving group. In some embodiments, coupling of the PNA monomer is performed on a plurality of sites on said array simultaneously.

In some embodiments, the leaving group is a halo. In some embodiments, the coupling further comprises displacing the leaving group of the acetic acid with a diamino-alkane, wherein one amine of the diamino-alkane is protected. In some embodiments, the diamino-alkane is ethylenediamine.

In some embodiments, the coupling further comprises activating a PNA monomer acetic acid by an activation agent, and coupling the activated PNA monomer acetic acid to the unprotected amine of the diamino-alkane. In some embodiments, the PNA monomer acetic acid is R-thymine-1-acetic acid, R-(cytosine-1-yl)-acetic acid, R-adenine-9-yl-acetic acid, R-guanine-9-acetic acid, or R-uracil-1-acetic acid, where R is H or a protection group.

Also provided herein is a method of analyzing a sample, said sample comprising nucleic acids obtained from a subject, comprising: contacting said sample with a PNA or PNA-DNA array described herein under conditions that promote hybridization between said sample and said array; detecting a signal from the array, wherein said signal indicates the presence, absence or amount of sample hybridized to said array at one or more of said feature locations; and analyzing said signal thereby analyzing the sample.

In some embodiments, analyzing the sample comprises determining a nucleic acid sequence present in said sample based on said signal. In some embodiments, analyzing the sample comprises determining the presence or absence of a SNP present in said sample based on said signal. In some embodiments, the array comprises PNA/DNA chimeras and the method further comprises carrying out a primer extension reaction following said hybridization between said sample and said array.

In some embodiments, the photoactive compound is about 0.5-5% by weight of the total formulation. In some embodiments, the photo-protective compound shields any compounds or coupling molecules attached to the microarray from the electromagnetic radiation. Examples of coupling molecules include, but are not limited to peptide nucleic acids (“PNA”) and the like. In some embodiments, the coupling molecule is 1-2% by weight of the total formulation. In some embodiments, the coupling molecule comprises a protected group. In some embodiments, the group is protected by Fmoc.

In some embodiments, each PNA or PNA-DNA chain is at least 6 monomers in length. In some embodiments, each PNA or PNA-DNA chain is at least 6, 10, 15, 20, 25, 30, 35, 40, 45, 50, 55, or 60 monomers in length. In some embodiments, each PNA or PNA-DNA chain comprises one or more L-chiral PNA monomers. In some embodiments, each PNA or PNA-DNA chain comprises one or more D-chiral PNA monomers. In some embodiments, each PNA or PNA-DNA chain comprises one or more synthetic PNA or DNA monomers. In some embodiments, the array comprises at least 1,000 different PNA or PNA-DNA chains attached to the surface. In some embodiments, the array comprises at least 10,000 different PNA or PNA-DNA chains attached to the surface.

In some embodiments, each of the positionally-defined locations is at a different, known location that is physically separated from each of the other positionally-defined locations. In some embodiments, each of the positionally-defined locations comprises a plurality of identical PNA or PNA-DNA sequences. In some embodiments, each positionally-defined location comprises a plurality of identical PNA sequences unique from the other positionally-defined locations. In some embodiments, each of the positionally-defined locations is a positionally-distinguishable location. In certain embodiments, each determinable PNA or PNA-DNA sequence corresponds to a known nucleotide sequence. In certain embodiments, each determinable PNA-DNA sequence is a distinct sequence. In some embodiments, the features are covalently attached to the surface. In some embodiments, PNA or PNA-DNA chains are attached to the surface through a linker molecule or a coupling molecule.

### BRIEF DESCRIPTION OF THE SEVERAL VIEWS OF THE DRAWINGS

These and other features, embodiments, and advantages of the present invention will become better understood with regard to the following description, and accompanying drawings, where:

FIG. 1 shows a method of manufacturing a PNA array, according to some embodiments.

FIG. 2A and 2B shows a method of manufacturing a PNA-DNA chimera array and a reaction scheme for selected steps, according to some embodiments.

FIG. 3 depicts an exemplary synthetic scheme for the reverse phosphoramidite approach.

FIG. 4 depicts a general synthetic scheme for adding custom or preselected oligonucleotides to a PNA oligomer.

FIG. 5 depicts phosphoramidite chemistry to access a sequence from the 3′ position of the donor nucleotide to the 3′ position of the acceptor nucleotide.

FIG. 6 shows fluorescein intensity to measure hybridization of a correct and mismatched sequence attached to a PNA molecule bound to an array, according to some embodiments.

FIG. 7 depicts an exemplary single nucleotide primer extension reaction to detect a sequence variant.

### DETAILED DESCRIPTION

Terms used in the claims and specification are defined as set forth below unless otherwise specified.

As used herein, the term “wafer” refers to a slice of semiconductor material, such as a silicon or a germanium crystal generally used in the fabrication of integrated circuits. Wafers can be in a variety of sizes from, e.g., 25.4 mm (1 inch) to 300 mm (11.8 inches) along one dimension with thickness from, e.g., 275 μm to 775 μm.

As used herein, the term “photomask” or “reticle” or “mask” refers to an opaque plate with transparent patterns or holes that allow light to pass through. In a typical exposing process, the pattern on a photomask is transferred onto a photoresist.

As used herein, the term “photoresist” or “resist” or “photoactive material” refers to a light-sensitive material that undergoes a chemical modification, e.g., changes its solubility in a solution or generates a photoacid, when exposed to electromagnetic radiation, in particular ultra violet or deep ultra violet radiation. Photoresists includes organic or inorganic compounds.

As used herein the term “photoresist formulation” refers to a formulation including a photoactive compound and a photo-protective compound.

As used herein, the term “photoactive compound” refers to compounds that are modified when exposed to electromagnetic radiation. These compounds include, for example, cationic photoinitiators such as photoacid generators (PAGs) or photobase generators (PBGs), which generate a corresponding photoacid or photobase, respectively, when exposed to electromagnetic radiation. Examples of photoactive compounds are disclosed in the International Patent Application No. PCT/US2013/070207, filed Nov. 14, 2013, which is incorporated herein in its entirety for all purposes. A photoinitiator is a compound especially added to a formulation to convert electromagnetic radiation into chemical energy in the form of initiating species, e.g., free radicals or cations. The acid, base, or other product of a photoactive compound exposed to electromagnetic radiation may then react with another compound in a chain reaction to produce a desired chemical reaction. The spatial orientation of the occurrence of these chemical reactions is thus defined according to the pattern of electromagnetic radiation the solution or surface comprising photoactive compounds is exposed to. This pattern may be defined, e.g., by a photomask or reticle.

As used herein, the term “photo-protective” compound refers to an organic or anorganic compound that shields electromagnetic radiation by absorbing or scattering the radiation so that the energy of the radiation beyond the photo-protective compound along the direction of the radiation is decreased. Examples of anorganic photo-protective compounds include, but are not limited to titanium dioxide, zinc sulfide, and magnesium fluoride

As used herein, the term “coupling molecule” or “monomer molecule” includes any natural or artificially synthesized peptide nucleic acid (PNA) or PNA monomer acetic acid with their nitrogens optionally protected with a fluorenylmethyloxycarbonyl (Fmoc or F-Moc) group or a t-butoxycarbonyl (tboc or Boc) group. Alternatively, these terms include substituted acetic acid with the substitution being a leaving group. Examples of coupling molecules include R-thymine-1-acetic acid, R-(cytosine-1-yl)-acetic acid, R-adenine-9-yl-acetic acid, R-guanine-9-acetic acid, and R-uracil-1-acetic acid, where R is H or a protection group for the nucleic acid monomer. Other examples are described below.

As used herein, the term “coupling” or “coupling process” or “coupling step” refers to a process of forming a bond between two or more molecules such as a linking molecule or a coupling molecule. A bond can be a covalent bond such as a peptide bond. A peptide bond is a chemical bond formed between two molecules when the carboxyl group of one coupling molecule reacts with the amino group of the other coupling molecule, releasing a molecule of water (H₂O). This is a dehydration synthesis reaction (also known as a condensation reaction), and usually occurs between amino acids. The resulting —C(═O)NH— bond is called a peptide bond, and the resulting molecule is an amide. As used herein, coupling a PNA monomer includes coupling a portion of a PNA or PNA precursor, then building up the PNA.

As used herein, the term “coupling efficiency” refers to the probability of successful addition of a monomer to a reaction site (e.g., at the end of a polymer) available for binding to the monomer. For example, during the growth of a PNA polymer (also referred to as PNA sequence) in the C to N orientation, a PNA monomer having a free carboxyl group would bind to another PNA monomer having a free amine group under appropriate conditions. The coupling efficiency gives the probability of the addition of a free carboxyl acid to the free amino group under certain conditions. It may be determined in bulk, e.g., by monitoring single monomer additions to several unique reaction sites simultaneously.

As used herein, the term “biomarkers” includes, but is not limited to PNA, DNA, RNA, proteins (e.g., enzymes such as kinases), peptides, sugars, salts, fats, lipids, ions and the like.

As used herein, the term “linker molecule” or “spacer molecule” includes any molecule that does not add any functionality to the resulting PNA monomer or PNA polymer or PNA-DNA chimeric polymer but spaces and extends the monomer or polymer out from the substrate, thus increasing the distance between the substrate surface and the growing PNA or PNA-DNA sequence. This generally reduces steric hindrance with the substrate for reactions involving PNA or PNA-DNA polymers (including uni-molecular folding reactions and multi-molecular binding reactions) and so improves performance of assays measuring one or more embodiments of PNA or PNA-DNA functionality.

As used herein, the term “developer” refers to a solution that can selectively dissolve the materials that are either exposed or not exposed to light. Typically developers are water-based solutions with minute quantities of a base added. Examples include tetramethyl ammonium hydroxide in water-based developers. Developers are used for the initial pattern definition where a commercial photoresist is used.

As used herein, the term “protecting group” includes a group that is introduced into a molecule by chemical modification of a functional group to obtain chemoselectivity in a subsequent chemical reaction. Chemoselectivity refers to directing a chemical reaction along a desired path to obtain a pre-selected product as compared to another. For example, the use of tboc as a protecting group enables chemoselectivity for PNA synthesis using a light mask and a photoacid generator to selectively remove the protecting group and direct pre-determined PNA coupling reactions to occur at locations defined by the light mask.

As used herein, the term “microarray,” “array” or “chip” refers to a substrate on which a plurality of probe molecules of specific PNA, RNA or DNA binding sequences have been affixed at separate locations in an ordered manner thus forming a microscopic array. Specific PNA, RNA or DNA binding sequences may be bound to the substrate of the chip through one or more different types of linker molecules. A “chip array” refers to a plate having a plurality of chips, for example, 24, 96, or 384 chips.

As used herein, the term “probe molecules” refers to, but is not limited to, peptide nucleic acids (“PNA”), DNA binding sequences, oligonucleotides, nucleic acids, deoxyribonucleic acids (DNA), ribonucleic acids (RNA), nucleotide mimetics, chelates, side-chain modified peptide sequences, biomarkers and the like. As used herein, the term “feature” refers to a particular probe molecule that has been attached to a microarray. As used herein, the term “ligand” refers to a molecule, agent, analyte or compound of interest that can bind to one or more features.

As used herein, the term “microarray system” or a “chip array system” refers to a system usually comprised of bio molecular probes formatted on a solid planar surface like glass, plastic or silicon chip plus the instruments needed to handle samples (automated robotics), to read the reporter molecules (scanners) and analyze the data (bioinformatic tools).

As used herein the term “patterned region” or “pattern” or “location” refers to a region on the substrate on which are grown different features. These patterns can be defined using photomasks.

As used herein, the term “derivatization” refers to the process of chemically modifying a surface to make it suitable for biomolecular synthesis. Typically, derivatization includes the following steps: making the substrate hydrophilic, adding an amino silane group, and attaching a linker molecule.

As used herein, the term “capping” or “capping process” or “capping step” refers to the addition of a molecule that prevents the further reaction of the molecule to which it is attached. For example, to prevent the further formation of a peptide bond, the amino groups are typically capped with an acetic anhydride molecule. In other embodiments, ethanolamine is used.

As used herein, the term “diffusion” refers to the spread of, e.g., photoacid or photobase through random motion from regions of higher concentration to regions of lower concentration.

As used herein, the term “dye molecule” refers to a dye which typically is a colored substance that can bind to a substrate. Dye molecules can be useful in detecting binding between a feature on an array and a molecule of interest.

As used herein, the terms “immunological binding” and “immunological binding properties” refer to the non-covalent interactions of the type which occur between an immunoglobulin molecule and an antigen for which the immunoglobulin is specific.

As used herein, the term “biological sample” refers to a sample derived from biological tissue or fluid that can be assayed for an analyte(s) of interest. Such samples include, but are not limited to, sputum, amniotic fluid, blood, blood cells (e.g., white cells), tissue or fine needle biopsy samples, urine, peritoneal fluid, and pleural fluid, or cells therefrom. Biological samples may also include sections of tissues such as frozen sections taken for histological purposes. Although the sample is typically taken from a human patient, the assays can be used to detect analyte(s) of interest in samples from any organism (e.g., mammal, bacteria, virus, algae, or yeast) or mammal, such as dogs, cats, sheep, cattle, and pigs. The sample may be pretreated as necessary by dilution in an appropriate buffer solution or concentrated, if desired.

As used herein, the term “assay” refers to a type of biochemical test that measures the presence or concentration of a substance of interest in solutions that can contain a complex mixture of substances.

The term “percent identity,” in the context of two or more nucleic acid or PNA sequences, refer to two or more sequences or subsequences that have a specified percentage of nucleobases that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

Unless otherwise noted, “alkyl” as used herein, whether used alone or as part of a substituent group, refers to a saturated, branched, or straight-chain monovalent hydrocarbon radical derived by the removal of one hydrogen atom from a single carbon atom of a parent alkane. Typical alkyl groups include, but are not limited to, methyl; ethyls; propyls such as propan-1-yl, propan-2-yl; butyls such as butan-1-yl, butan-2-yl, 2-methyl-propan-1-yl, 2-methyl-propan-2-yl, and the like. In preferred embodiments, the alkyl groups are C₁₋₆ alkyl, with C₁₋₃ alkyl being particularly preferred. “Alkoxyl” radicals are oxygen ethers formed from the previously described straight or branched chain alkyl groups.

As used herein, “halo” or “halogen” shall mean chlorine, bromine, fluorine and iodine. “Halo substituted” shall mean a group substituted with at least one halogen atom, preferably substituted with a least one fluoro atom. Suitable examples include, but are not limited to —CF₃, and the like.

The term “cycloalkyl,” as used herein, refers to a stable, saturated or partially saturated monocyclic or bicyclic ring system containing from 3 to 8 ring carbons and preferably 5 to 7 ring carbons. Examples of such cyclic alkyl rings include cyclopropyl, cyclobutyl, cyclopentyl, cyclohexyl or cycloheptyl.

The term “alkenyl” refers to an unsaturated branched, straight-chain or cyclic monovalent hydrocarbon radical, which has at least one carbon-carbon double bond, derived by the removal of one hydrogen atom from a single carbon atom of a parent alkene. The radical may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

The term “heteroaryl” refers to a monovalent heteroaromatic radical derived by the removal of one hydrogen atom from a single atom of a parent heteroaromatic ring system. Typical heteroaryl groups include monocyclic and bicyclic systems where one or both rings is heteroaromatic. Heteroaromatic rings may contain 1-4 heteroatoms selected from O, N, and S. Examples include but are not limited to, radicals derived from carbazole, furan, imidazole, indazole, indole, indolizine, isoindole, isoquinoline, isothiazole, isoxazole, naphthyridine, oxadiazole, oxazole, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

The term “aryl,” as used herein, refers to aromatic groups comprising a stable six-membered monocyclic, or ten-membered bicyclic or fourteen-membered tricyclic aromatic ring system, which consists of carbon atoms. Examples of aryl groups include, but are not limited to, phenyl or naphthalenyl.

The term “heterocyclyl” is a 3- to 12-member saturated or partially saturated single (monocyclic), bicyclic, or fused ring system which consists of carbon atoms and from 1 to 6 heteroatoms selected from N, O and S. The heterocyclyl group may be attached at any heteroatom or carbon atom which results in the creation of a stable structure. The bicyclic heterocyclyl group includes systems where one or both rings include heteroatoms. Examples of heterocyclyl groups include, but are not limited to, 2-imidazoline, imidazolidine; morpholine, oxazoline, oxazolidine, 2-pyrroline, 3-pyrroline, pyrrolidine, pyridone, pyrimidone, piperazine, piperidine, indoline, tetrahydrofuran, 2-pyrroline, 3-pyrroline, 2-imidazoline, 2-pyrazoline, indolinone, thiomorpholine, tetrahydropyran, tetrahydroquinoline, tetrahydroquinazoline, [1,2,5]thiadiazolidine 1,1-dioxide, [1,2,3]oxathiazolidine 2,2-dioxide, and the like.

The term “cis-trans isomer” refers to stereoisomeric olefins or cycloalkanes (or hetero-analogues) which differ in the positions of atoms (or groups) relative to a reference plane: in the cis-isomer the atoms of highest priority are on the same side; in the trans-isomer they are on opposite sides.

The term “substituted” refers to a radical in which one or more hydrogen atoms are each independently replaced with the same or different substituent(s).

With reference to substituents, the term “independently” means that when more than one of such substituent is possible, such substituents may be the same or different from each other.

The term “oxo” whether used alone or as part of a substituent group refers to an O═ bounded to either a carbon or a sulfur atom. For example, phthalimide and saccharin are examples of compounds with oxo substituents.

The term “PNA-DNA chimera” refers to an oligomer, or oligomers, comprised of: (i) a contiguous moiety of PNA monomer units and (ii) a contiguous moiety of nucleotide monomer units with an enzymatically-extendable terminus

The term “primer extension” refers to an enzymatic addition, i.e., polymerization, of monomeric nucleotide units to a primer while the primer is hybridized (annealed) to a template nucleic acid.

It must be noted that, as used in the specification and the appended claims, the singular forms “a,” “an,” and “the” include plural referents unless the context clearly dictates otherwise.

### Compositions and Synthetic Schemes

### PNA Synthesis on Microarray

This application provides methods of making peptide nucleic acid (PNA) compounds, including PNA monomers and polymers, and methods of making PNA-DNA chimera on a microarray platform through high throughput parallel synthesis using photolithography. Some embodiments use UV radiation at 248 nm for making PNA or PNA-DNA chimera microarrays.

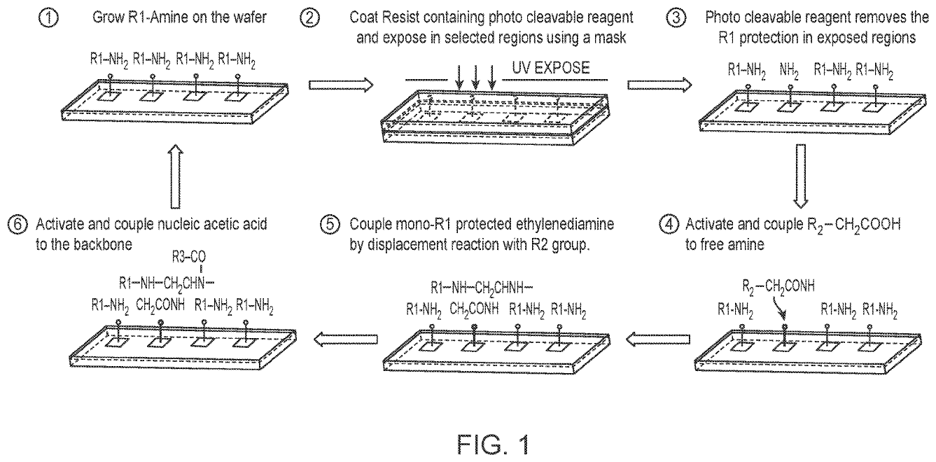

FIG. 1 and Scheme 1 illustrate the suggested synthetic routes, according to some embodiments, for PNA oligomer synthesis. Using the scheme, the guidelines below, and the examples, a person of skill in the art may develop analogous or similar methods for a given PNA compound or microarray having a plurality of PNA compounds that is within the scope of the invention. In some embodiments, the PNA compounds are coupled to the microarray in positionally-defined locations. These methods are representative of the synthetic schemes, but are not to be construed as limiting the scope of the invention.

In some embodiments, as illustrated in FIG. 1, the method includes providing a microarray wafer having surface that includes attached protected amine groups (FIG. 1, Part No. 1). The protection group of the amine is R₁, which, in some embodiments, includes Carbobenzyloxy (Cbz), p-Methoxybenzyl carbonyl (Moz), tert-Butyloxycarbonyl (Boc), Carbamate group, Methoxytrityl (Mmt), Dimethoxytrityl (DMT), Trityl (Trt), Tosyl (Ts), tert-amyloxycarbonyl, adamantyloxycarbonyl, 1-methylcyclobutyloxycarbonyl, 2-(p-biphenyl)propyl(2)oxycarbonyl, 2-(p-phenylazophenylyl)propyl(2) oxycarbonyl, alpha,alpha-dimethyl-3,5-dimethyloxybenzyloxy-carbonyl, 2-phenylpropyl(2)oxycarbonyl, 4-methyloxybenzyloxycarbonyl, furfuryloxycarbonyl, p-toluenesulfenylaminocarbonyl, dimethylphosphinothioyl, diphenylphosphinothioyl, 2-benzoyl-1-methylvinyl, o-nitrophenylsulfenyl, and 1-naphthylidene, all of which are acid labile. In some embodiments, the protection group of the amine is Acetyl (Ac), Benzoyl (Bz), 9-Fluorenylmethyloxycarbonyl (Fmoc), methylsulfonylethyloxycarbonyl, and 5-benzisoazolylmethyleneoxycarbonyl, which are generally base labile.

In some embodiments, the wafer of the microarray is spin-coated with a photoresist formulation (FIG. 1, Part No. 2). In some embodiments, the photoresist formulation includes a photoactive compound and a photo-protective compound. The photoactive compound is also referred to as a photocleavable reagent. In some embodiments, the photoactive compound is a photoacid generator (PAG) or a photobase generator (PBG). In some embodiments, the polymer is poly(vinyl alcohol), dextran, sodium alginate, poly(aspartic acid), poly(ethylene glycol), poly(ethylene oxide), poly(vinyl pyrrolidone), poly(acrylic acid), poly(acrylic acid)-sodium salt, poly(acrylamide), poly(N-isopropyl acrylamide), poly(hydroxyethyl acrylate), poly(acrylic acid), poly(sodium styrene sulfonate), poly(-acrylamido-2-methyl-1-propanesulfonic acid), polysaccharides, and cellulose derivatives. In some embodiments, the photo-protective compound is titanium dioxide, zinc sulfide, and magnesium fluoride.

In some embodiments, the method includes forming a photoresist layer on the wafer surface by applying the photoresist formulation. The photoresist layer shields groups or compounds, including PNA compounds, attached to the microarray from radiation, to which the wafer is exposed. In some embodiments, the shielded groups or compounds are included in a layer that is located between the wafer surface and the photoresist layer (FIG. 1, Part No. 2).

In some embodiments, the photoresist formulation further includes a polymer and a solvent. In some embodiments, the solvent is water, an organic solvent, or a combination thereof. In some embodiments, the organic solvent is N-methyl pyrrolidone, dimethyl formamide, dichloromethane, dimethyl sulfoxide, propylene glycol methyl ether acetate (PGMEA), ethyl lactate, ethoxyethyl acetate, or a combination thereof.

In some embodiments, the wafer is spin-coated in the range of 2000 rpm to 4000 rpm, preferably in the range of 2500 rpm to 3000 rpm, for 10-180 seconds, preferably for 60-120 seconds, with the photoresist formulation. Following the spin-coating of the photoresist formulation, the wafer is exposed to radiation according to a pattern defined by a photomask, wherein the locations exposed to the radiation undergo acid or base generation due to the presence of the photo acid generator or photo base generator in the photoresist formulation. In some embodiments, the radiation includes 248 nm ultraviolet light in a deep ultra violet scanner tool. In some embodiments, the radiation includes 365 nm ultraviolet light. Radiation to activate a photo acid or photo base generator can be in a range of wavelengths, and is not limited to wavelengths disclosed herein. In some embodiments, the expose energy is between 1 mJ/cm² to 100 mJ/cm², preferably in the range of 30-60 mJ/cm².

In some embodiments, the surface of the wafer is post-baked after exposure to the radiation in a bake module. In some embodiments, the post bake temperature varies between 75° Celsius to 115° Celsius, for about 60 seconds, typically not exceeding 180 seconds.

In some embodiments, upon radiation exposure, the photoacid generator or photobase generator yields a photoacid or photobase, which in turn removes the protection of amino groups in the exposed regions on the wafer surface of the microarray (Step 1 in Scheme 1), followed by stripping the photoresist layer (FIG. 1, Part No. 3). In some embodiments, the yielded photoacid or photobase (also referred to as reacted photo cleavable reagent) diffuses from the photoresist layer to the layer that includes the shielded groups or compounds, e.g., the PNA compounds attached to the microarray surface.

In some embodiments, the unprotected (free) amino groups are coupled to an activated R₂-acetic acid by spin coating an activating formulation including R₂-acetic acid and an activation agent, and reacted in a bake module with the temperature varying from 55° Celsius to 115° Celsius for 60-240 seconds (Step 2 in Scheme 1; Fig., Part No. 4). In some embodiments, R₂ is a leaving group. In some embodiments, the leaving group is bromo, chloro, fluoro, iodo, or the like. In some embodiments, R₂-acetic acid is bromoacetic acid, chloroacetic acid, fluoroacetic acid, iodoacetic acid, or the like.

In some embodiments, the activation agent is 1-ethyl-3-(3-dimethyl-aminopropyl)-carbodiimide (EDC), N-hydroxysuccinimide (NHS), 1,3-diisopropyl-carbodiimide (DIC), hydroxybenzotriazole (HOBt), O-(7-azabenzotriazol-1-yl)-N,N,N′,N′-tetramethyluronium hexafluorophosphate (HATU), benzotriazol-1-yl-oxytripyrrolidinophosphonium hexafluorophosphate (PyBOP), N,N-diisopropylethylamine (DIEA), N-hydroxysuccinimide (HOSu), N-hydroxy-5-norbornene-2,3-dicarboximide (HONB), 6-chloro-1-hydroxybenzotriazole (6-Cl-HOBt), 3-hydroxy-4-oxo-3,4-dihydro-1,2,3-benzotriazine (HODhbt) and its aza derivative (HODhat), or any combination thereof.

In some embodiments, the concentration of the R₂-acetic acid is less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0 weight % of the total formulation concentration.

In some embodiments, the concentration of the activation agent is less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0 weight % of the total formulation concentration.

In some embodiments, the coupling of the activated R₂-acetic acid is followed by displacement reaction with a mono-amino protected ethylenediamine (also referred to as mono-R₁ protected ethylenediamine) with a protecting group R₁ (Step 3 in Scheme 1; FIG. 1, Part No. 5). In some embodiments, the displacement reaction includes a mono-amino protected diamino-alkane. “Mono-amino protected” refers to only one of the two amino groups being protected. In some embodiments, a displacement formulation that includes the mono-R₁ protected ethylenediamine is spin coated on the wafer and reacted in a bake module with the temperature varying from 55° Celsius to 115° Celsius for 30-300 seconds, preferably for 120 seconds.

In some embodiments, the protection group R₁ is any of the above described amino protection group. In some embodiments, the concentration of the mono-amino protected ethylenediamine is less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0 weight % of the total formulation concentration.

In some embodiments, the displacement reaction is followed by a coupling reaction with a PNA monomer acetic acid (Step 4 in Scheme 1; FIG. 1, Part No. 6). In some embodiments, the PNA monomer acetic acid is R₃-acetic acid, including, but not limited to R-thymine-1-acetic acid, R-(cytosine-1-yl)-acetic acid, R-adenine-9-yl-acetic acid, R-guanine-9-acetic acid, and R-uracil-1-acetic acid, where R is H or a protection group for the nucleic acid monomer and, in some embodiments, is Boc, Bis-Boc, Alloc, Benzoyl, Acetyl, Fmoc, Trityl, any of the above described amino protection group. In some embodiments, the PNA monomer acetic acid is activated with an activation agent prior to coupling to the displaced amine. In some embodiments, the wafer is spin-coated with an activating formulation including the PNA monomer acetic acid and the activation agent, and reacted for 90-300 seconds.

In some embodiments, the activation agent is 1-ethyl-3-(3-dimethyl-aminopropyl)-carbodiimide (EDC), N-hydroxysuccinimide (NHS), 1,3-diisopropyl-carbodiimide (DIC), hydroxybenzotriazole (HOBt), O-(7-azabenzotriazol-1-yl)-N,N,N′,N′-tetramethyluronium hexafluorophosphate (HATU), benzotriazol-1-yl-oxytripyrrolidinophosphonium hexafluorophosphate (PyBOP), N,N-diisopropylethylamine (DIEA), N-hydroxysuccinimide (HOSu), N-hydroxy-5-norbornene-2,3-dicarboximide (HONB), 6-chloro-1-hydroxybenzotriazole (6-Cl-HOBt), 3-hydroxy-4-oxo-3,4- dihydro-1,2,3-benzotriazine (HODhbt) and its aza derivative (HODhat), or any combination thereof.

In some embodiments, the wafer surface is optionally spin-coated with a capping solution to prevent the non-reacted amino groups on the wafer from reacting to the next coupling molecule, e.g., the activated R₂-acetic acid. In some embodiments, the capping solution includes a capping molecule, a solvent, a polymer, and a coupling molecule. In some embodiments, the solvent is an organic solvent like N-methyl pyrrolidone, dimethyl formamide, or combinations thereof. In some embodiments, the capping molecule is an acetic anhydride and the polymer is polyvinyl pyrrolidone, polyvinyl alcohol, polymethyl methacrylate, poly-(methyl-isopropenyl)-ketone, or poly-(2-methyl-pentene-1-sulfone). In some embodiments, the capping solution is spin-coated on the wafer at 1500-3500 rpm for at least 30 seconds and reacted in a bake module with the temperature varying from 55° Celsius to 95° Celsius for 30-90 seconds, preferably 60 seconds to complete one cycle.

In some embodiments, the entire synthesis cycle (FIG. 1, Part 1-6) is repeated with particular PNA monomer acetic acids in each cycle to yield a specific PNA sequence of a desired length.

Examples of the described synthetic cycle is further illustrated in Scheme 1 and Examples 1 through 8. Compounds analogous to the target compounds of these examples can be made according to similar synthesis routes. The disclosed compounds are useful in the manufacture of microarrays as described herein. The wavy line ( [symbol] ) in the disclosed Schemes indicates the coupling of the particular compound to the surface of the microarray.

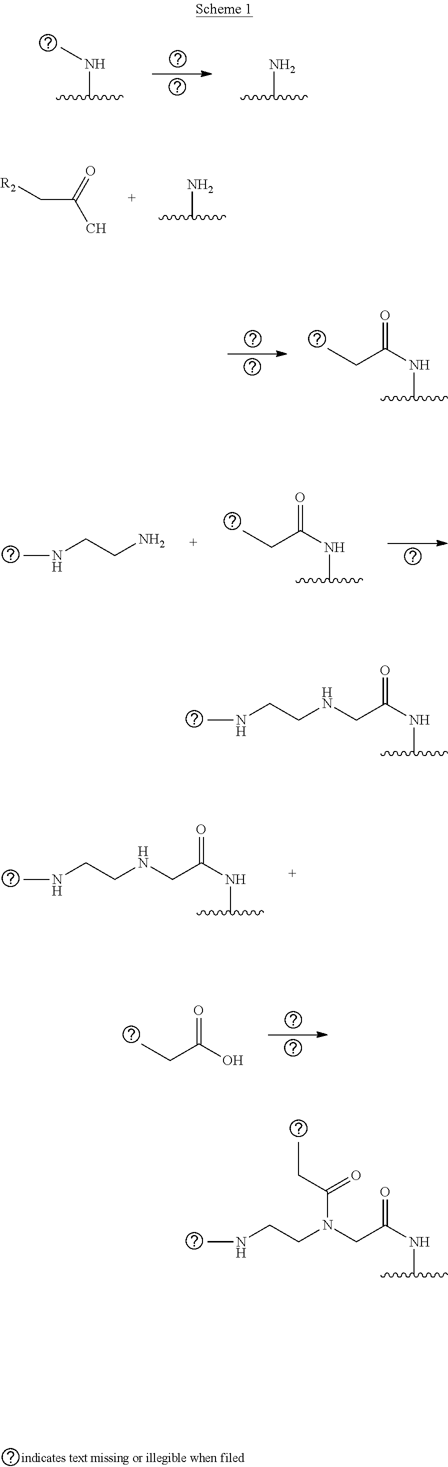

Where the compounds according to this invention have at least one chiral center, they may accordingly exist as enantiomers. Where the compounds possess two or more chiral centers, they may additionally exist as diastereomers. Where the processes for the preparation of the compounds according to the invention give rise to mixtures of stereoisomers, these isomers may be separated by techniques such as preparative chromatography. The compounds may be prepared in racemic form or as individual enantiomers or diastereomers by either stereospecific synthesis or by resolution. The compounds may, for example, be resolved into their component enantiomers or diastereomers by techniques, such as the formation of stereoisomeric pairs by salt formation with an optically active base, followed by fractional crystallization and regeneration of the free acid. The compounds may also be resolved by formation of stereoisomeric esters or amides, followed by chromatographic separation and removal of the chiral auxiliary. Alternatively, the compounds may be resolved using a chiral HPLC column. It is to be understood that all stereoisomers, racemic mixtures, diastereomers, geometric isomers, and enantiomers thereof are encompassed within the scope of the present invention.

Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

### DNA Monomer Couplink to PNA on a Microarray

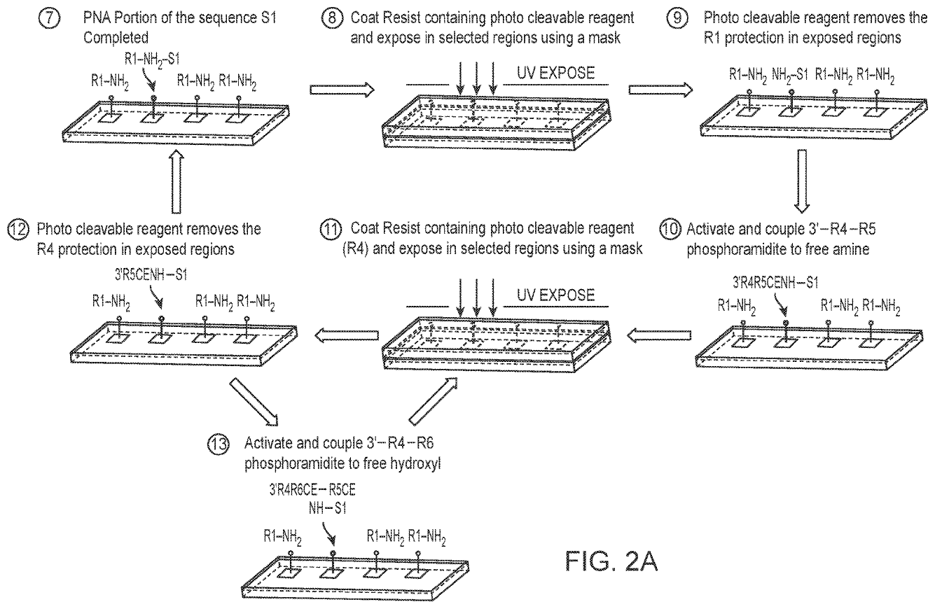

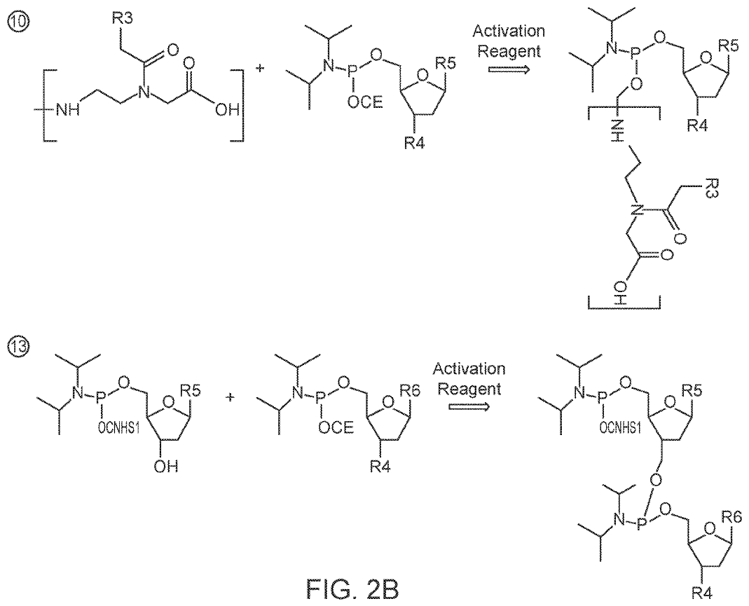

FIG. 1 and FIG. 2A and 2B and illustrate the suggested synthetic routes, according to some embodiments, for PNA-DNA chimera oligomer synthesis. Using the scheme, the guidelines below, and the examples, a person of skill in the art may develop analogous or similar methods for a given PNA-DNA chimera or microarray having a plurality of PNA-DNA chimera that is within the scope of the invention. In some embodiments, the PNA-DNA chimera are coupled to the microarray in positionally-defined locations. These methods are representative of the synthetic schemes, but are not to be construed as limiting the scope of the invention.

In some embodiments PNA-DNA chimera are synthesized through three distinct types of covalent binding (i.e., covalent attachment): i) covalent binding of a PNA monomer to another PNA monomer, a PNA oligomer, or a free amine affixed to the surface of the array; ii) covalent binding of a DNA monomer to a PNA monomer at the end of a PNA oligomer affixed to the surface of the array; or iii) covalent binding of a DNA monomer to a DNA monomer at the end of a PNA-DNA chimeric oligomer. Covalent binding of a PNA monomer is as described above.

FIG. 2A and 2B illustrate the suggested synthetic routes, according to some embodiments, for covalent attachment of a DNA monomer to a PNA oligomer (steps 7-10) and covalent attachment of a DNA monomer to the 5′ end of a PNA-DNA chimeric oligomer (steps 11-13).

In some embodiments, as illustrated in FIG. 2A, the method includes providing a microarray wafer having surface that includes attached protected amine groups at end of a PNA oligomer (FIG. 2A, Part No. 7). Examples of protection groups suitable for protection are provided herein. In some embodiments the PNA oligomer is synthesized on an array according the methods provided herein.

In some embodiments, the wafer of the microarray is spin-coated with a photoresist formulation (FIG. 2A, Part No. 8). In some embodiments, the photoresist formulation includes a photoactive compound and a photo-protective compound. The photoactive compound is also referred to as a photocleavable reagent. In some embodiments, the photoactive compound is a photoacid generator (PAG) or a photobase generator (PBG). In some embodiments, the polymer is poly(vinyl alcohol), dextran, sodium alginate, poly(aspartic acid), poly(ethylene glycol), poly(ethylene oxide), poly(vinyl pyrrolidone), poly(acrylic acid), poly(acrylic acid)-sodium salt, poly(acrylamide), poly(N-isopropyl acrylamide), poly(hydroxyethyl acrylate), poly(acrylic acid), poly(sodium styrene sulfonate), poly(-acrylamido-2-methyl-1-propanesulfonic acid), polysaccharides, and cellulose derivatives. In some embodiments, the photo-protective compound is titanium dioxide, zinc sulfide, and magnesium fluoride.

In some embodiments, the method includes forming a photoresist layer on the wafer surface by applying the photoresist formulation. The photoresist layer shields groups or compounds, including PNA compounds, attached to the microarray from radiation, to which the wafer is exposed. In some embodiments, the shielded groups or compounds are included in a layer that is located between the wafer surface and the photoresist layer (FIG. 1, Part No. 2).

In some embodiments, the photoresist formulation further includes a polymer and a solvent. In some embodiments, the solvent is water, an organic solvent, or a combination thereof. In some embodiments, the organic solvent is N-methyl pyrrolidone, dimethyl formamide, dichloromethane, dimethyl sulfoxide, propylene glycol methyl ether acetate (PGMEA), ethyl lactate, ethoxyethyl acetate, or a combination thereof.

In some embodiments, the wafer is spin-coated in the range of 2000 rpm to 4000 rpm, preferably in the range of 2500 rpm to 3000 rpm, for 10-180 seconds, preferably for 60-120 seconds, with the photoresist formulation. Following the spin-coating of the photoresist formulation, the wafer is exposed to radiation according to a pattern defined by a photomask, wherein the locations exposed to the radiation undergo acid or base generation due to the presence of the photo acid generator or photo base generator in the photoresist formulation. In some embodiments, the radiation includes 248 nm ultraviolet light in a deep ultra violet scanner tool. In some embodiments, the radiation includes 365 nm ultraviolet light. Radiation to activate a photo acid or photo base generator can be in a range of wavelengths, and is not limited to wavelengths disclosed herein. In some embodiments, the expose energy is between 1 mJ/cm² to 100 mJ/cm², preferably in the range of 30-60 mJ/cm².

In some embodiments, the surface of the wafer is post-baked after exposure to the radiation in a bake module. In some embodiments, the post bake temperature varies between 75° Celsius to 115° Celsius, for about 60 seconds, typically not exceeding 180 seconds.

In some embodiments, upon radiation exposure, the photoacid generator or photobase generator yields a photoacid or photobase, which in turn removes the protection of amino groups in the exposed regions on the wafer surface of the microarray, followed by stripping the photoresist layer (FIG. 2A, Part No. 9). In some embodiments, the yielded photoacid or photobase (also referred to as reacted photo cleavable reagent) diffuses from the photoresist layer to the layer that includes the shielded groups or compounds, e.g., the PNA compounds attached to the microarray surface.

In some embodiments, the unprotected (free) amino groups are coupled to an activated reverse phosphoamidite by spin coating an activating formulation including the reverse phosphoamidite and an activation agent, and reacted in a bake module with the temperature varying from 75° Celsius to 115° Celsius for 60-180 seconds (FIG. 2A and 2B, Part No. 10). In some embodiments, a reverse phosphoramidite comprises a dimethyoxytrityl (“DMT”) or similar protecting group at the 3′-hydroxyl of a deoxynucloeside. In some embodiments, the reverse amidite is 3′-R4-R5 phosphoramidite. In some embodiments, the activation agent is a tetrazole catalyst.

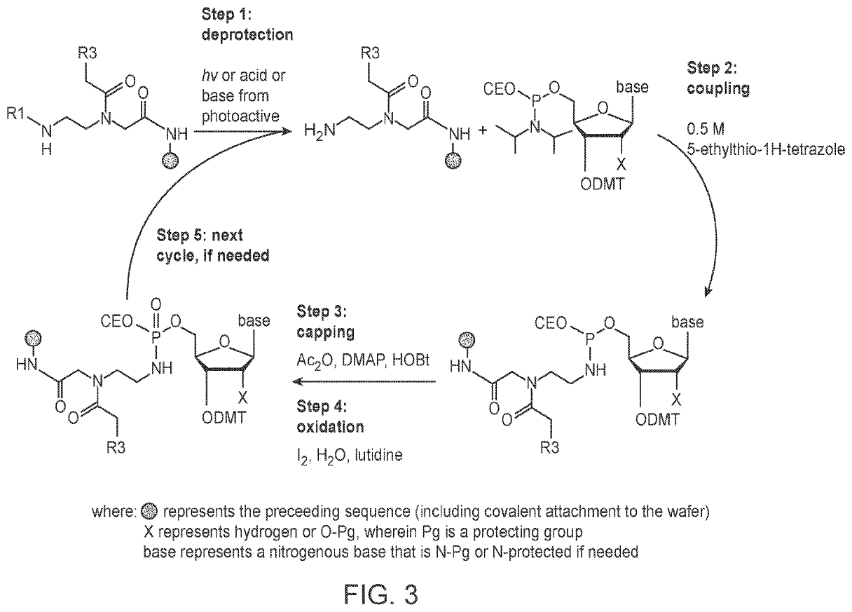

The exemplary synthetic scheme provided in FIG. 3 and described below provides the afore mentioned “reverse” (i.e. coupling to the 5′ position of the growing oligomer) phosphoramidite approach.

In some embodiments, a linker or a peptide/nucleotide can be covalently attached to a nucleotide through phosphoester routes (including using phosphodiester, phosphotriester, phosphite triester, phosphorothioate activated groups). Although dimethoxytrityl (DMT) is used in these examples, other protecting groups are well known to those of ordinary skill in the art. For example, one could use the phosphoramidite chemistry described above and a 5′-O-(α-methyl-6-nitropiperonyloxycarbonyl) (MeNPOC) protecting group at the 5′ or 3′ position of any given nucleotide. Such variations and adaptations with common protecting or “blocking” groups are within the ability of the skilled artisan to employ.

In some embodiments, the concentration of the reverse amidite is less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0 weight % of the total formulation concentration.

In some embodiments, the concentration of the activation agent is less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0 weight % of the total formulation concentration.

In some embodiments, the wafer surface is optionally spin-coated with a capping solution to prevent the non-reacted amino groups on the substrate from reacting to the next coupling molecule. The capping solution can be prepared as follows: a solvent, a polymer, and a coupling molecule. The capping solution is spin-coated on the wafer at 1500-3500 rpm for at least 30 seconds and reacted in a bake module with the temperature varying from 55° Celsius to 95° Celsius for 30 seconds-90 seconds preferably 60 seconds to complete one cycle. The phosphite-triester formed in the coupling step is then converted to a stable form which is achieved by iodine oxidation in the presence of water and pyridine.

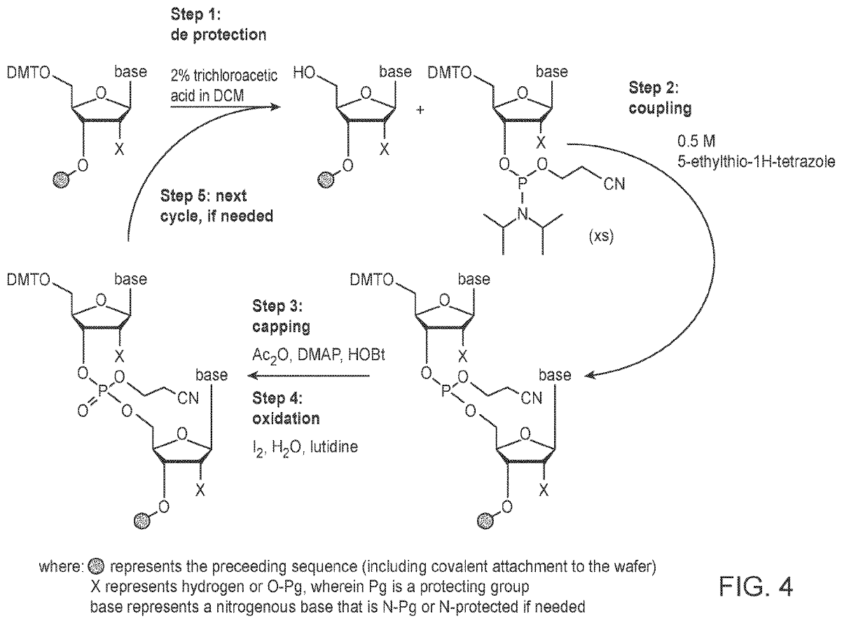

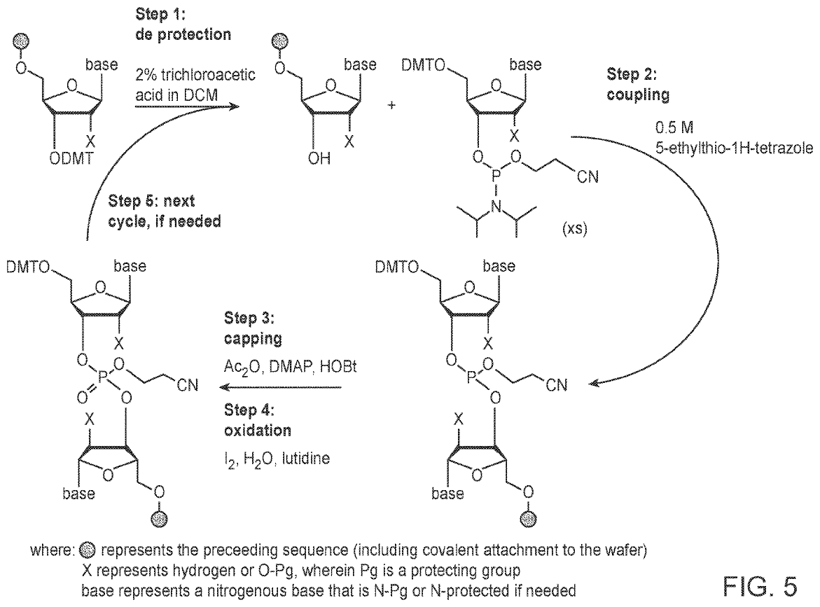

In some embodiments, the general synthetic scheme for adding custom or preselected oligonucleotides to a sequence (otherwise known in the art as oligonucleotide syntheses) is described in an example synthesis as shown in FIG. 4. As the skilled artisan will appreciate, this synthesis adds a nucleotide through the 5′ position of the donor nucleotide to the 3′ position of the acceptor nucleotide. In some embodiments, one can use the same phosphoramidite chemistry to access a sequence from the 3′ position of the donor nucleotide to the 3′ position of the acceptor nucleotide, as is shown in FIG. 5.

Accordingly, in some embodiments, one of skill in the art can mix and match the DMT, or otherwise known protecting groups and phosphoramidate activating groups on the 5′ and 3′ positions of the acceptor and donor nucleotides to couple the donor and acceptor together as desired, i.e. to produce any combination through the 5′ position as well. Thus, an amino acid of the presently disclosed PNA can be coupled to a nucleic acid according the several embodiments of the methods as described herein.

### DNA Monomer Couplink to PNA-DNA Chimeric Oligo on a Microarray

In some embodiments, as illustrated in FIG. 2A and 2B, the method includes providing a microarray wafer having surface that includes attached protected carboxylic acid groups at 5′ end of a DNA monomer on a PNA-DNA chimeric oligomer (FIG. 2A and 2B, Part No. 10 or 13). Examples of protection groups suitable for protection are provided herein. In some embodiments the PNA-DNA chimeric oligomer is synthesized on an array according the methods provided herein.

In some embodiments, the wafer of the microarray is spin-coated with a photoresist formulation (FIG. 2A, Part No. 11). In some embodiments, the photoresist formulation includes a photoactive compound and a photo-protective compound. The photoactive compound is also referred to as a photocleavable reagent. In some embodiments, the photoactive compound is a photoacid generator (PAG) or a photobase generator (PBG). In some embodiments, the polymer is poly(vinyl alcohol), dextran, sodium alginate, poly(aspartic acid), poly(ethylene glycol), poly(ethylene oxide), poly(vinyl pyrrolidone), poly(acrylic acid), poly(acrylic acid)-sodium salt, poly(acrylamide), poly(N-isopropyl acrylamide), poly(hydroxyethyl acrylate), poly(acrylic acid), poly(sodium styrene sulfonate), poly(-acrylamido-2-methyl-1-propanesulfonic acid), polysaccharides, and cellulose derivatives. In some embodiments, the photo-protective compound is titanium dioxide, zinc sulfide, and magnesium fluoride.

In some embodiments, the method includes forming a photoresist layer on the wafer surface by applying the photoresist formulation. The photoresist layer shields groups or compounds, including PNA compounds, attached to the microarray from radiation, to which the wafer is exposed. In some embodiments, the shielded groups or compounds are included in a layer that is located between the wafer surface and the photoresist layer (FIG. 1, Part No. 11).

In some embodiments, the photoresist formulation further includes a polymer and a solvent. In some embodiments, the solvent is water, an organic solvent, or a combination thereof. In some embodiments, the organic solvent is N-methyl pyrrolidone, dimethyl formamide, dichloromethane, dimethyl sulfoxide, propylene glycol methyl ether acetate (PGMEA), ethyl lactate, ethoxyethyl acetate, or a combination thereof.

In some embodiments, the wafer is spin-coated in the range of 2000 rpm to 4000 rpm, preferably in the range of 2500 rpm to 3000 rpm, for 10-180 seconds, preferably for 60-120 seconds, with the photoresist formulation. Following the spin-coating of the photoresist formulation, the wafer is exposed to radiation according to a pattern defined by a photomask, wherein the locations exposed to the radiation undergo acid or base generation due to the presence of the photo acid generator or photo base generator in the photoresist formulation. In some embodiments, the radiation includes 248 nm ultraviolet light in a deep ultra violet scanner tool. In some embodiments, the radiation includes 365 nm ultraviolet light. Radiation to activate a photo acid or photo base generator can be in a range of wavelengths, and is not limited to wavelengths disclosed herein. In some embodiments, the expose energy is between 1 mJ/cm² to 100 mJ/cm², preferably in the range of 30-60 mJ/cm².

In some embodiments, the surface of the wafer is post-baked after exposure to the radiation in a bake module. In some embodiments, the post bake temperature varies between 75° Celsius to 115° Celsius, for about 60 seconds, typically not exceeding 180 seconds.

In some embodiments, upon radiation exposure, the photoacid generator or photobase generator yields a photoacid or photobase, which in turn removes the protection of amino groups in the exposed regions on the wafer surface of the microarray, followed by stripping the photoresist layer (FIG. 2A, Part No. 12). In some embodiments, the yielded photoacid or photobase (also referred to as reacted photo cleavable reagent) diffuses from the photoresist layer to the layer that includes the shielded groups or compounds, e.g., the PNA-DNA chimeric oligo compounds attached to the microarray surface.

In some embodiments, the unprotected (free) carboxylic acid groups are coupled to an activated reverse phosphoramidite by spin coating an activating formulation including a reverse phosphoramidite and an activation agent, and reacted in a bake module with the temperature varying from 75° Celsius to 115° Celsius for 60-180 seconds (FIG. 2A and 2B, Part No. 13). In some embodiments, the reverse amidite is 3′-R4-R6 phosphoramidite. In some embodiments, the activation agent is a tetrazole catalyst.

In some embodiments, the concentration of the 3′-R4-R6 phosphoramidite is less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0 weight % of the total formulation concentration.

In some embodiments, the concentration of the activation agent is less than 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0 weight % of the total formulation concentration.

In some embodiments, the wafer surface is optionally spin-coated with a capping solution to prevent the non-reacted amino groups on the substrate from reacting to the next coupling molecule. The capping solution can be prepared as follows: a solvent, a polymer, and a coupling molecule. The capping solution is spin-coated on the wafer at 1500-3500 rpm for at least 30 seconds and reacted in a bake module with the temperature varying from 55° Celsius to 95° Celsius for 30 seconds-90 seconds preferably 60 seconds to complete one cycle. The phosphite-triester formed in the coupling step is then converted to a stable form which is achieved by iodine oxidation in the presence of water and pyridine.

In some embodiments, the entire DNA synthesis cycle (FIG. 2A and 2B, Part 11-13) is repeated with particular 3′-R4-R6 phosophramidites in each cycle to yield a specific DNA sequence of a desired length at the end of a PNA-DNA chimeric oligomer.

### Formulations

Disclosed herein are formulations such as photoresist formulations, displacement formulations, activating formulations, and linker formulations. These formulations can be useful in the manufacture and/or use of, e.g., PNA micoarrays disclosed herein. Generally, the components of each formulation disclosed herein are soluble in water at room temperature (app. 25° Celsius).

### Photoresist Formulations

Disclosed herein are photoresist formulations. In some embodiments, a photoresist formulation includes components such as a photoactive compound (also referred to as a photo cleavable reagent) and a photo-protective compound. In some embodiments, the photoactive compound is a photo acid generator or a photo base generator. In some embodiments, a photoresist formulation further includes a polymer and a solvent.

In some embodiments, the polymer is poly(vinyl alcohol), dextran, sodium alginate, poly(aspartic acid), poly(ethylene glycol), poly(ethylene oxide), poly(vinyl pyrrolidone), poly(acrylic acid), poly(acrylic acid)-sodium salt, poly(acrylamide), poly(N-isopropyl acrylamide), poly(hydroxyethyl acrylate), poly(acrylic acid), poly(sodium styrene sulfonate), poly(-acrylamido-2-methyl-1-propanesulfonic acid), polysaccharides, and cellulose derivatives. In some embodiments, the photo-protective compound is titanium dioxide, zinc sulfide, magnesium fluoride, and the like.

In one aspect, the photoresist formulation includes a photoactive compound. Photoactive compounds may include photobase generators or photoacid generators. Exposure of the photoactive compounds to electromagnetic radiation is a primary photochemical event that produces a compound that goes on to induce material transforming secondary reactions within a diffusion-limited radius. A photoresist formulation may comprise a photoactive compound comprising a radiation-sensitive catalyst precursor, e.g., a photoacid generator (PAG); a plurality of chemical groups that can react by elimination, addition, or rearrangement in the presence of catalyst; and optional additives to improve performance or processability, e.g., surfactants, photosensitizers, and etch resistors.

In some embodiments, a photoresist formulation includes a photobase generator and a photo sensitizer in a polymer matrix dispersed in a solvent. In some embodiments, the polymer in the composition of the photoresist is generally inert and non-crosslinking but the photoactive compounds will readily generate sufficient quantities of photobase upon exposure to electromagnetic radiation to bring about a desired reaction to produce a product at acceptable yield.

In some embodiments, a photoresist formulation can include various components such as a photosensitizer, a photoactive compound, a polymer, and a solvent.

In some embodiments, a photoactive compound can be a photoacid generator (PAG) or a photobase generator (PBG). Photoacid generators (or PAGs) are cationic photoinitiators. A photoinitiator is a compound especially added to a formulation to convert absorbed light energy, UV or visible light, into chemical energy in the form of initiating species, e.g., free radicals or cations. Cationic photoinitiators are used extensively in optical lithography. The ability of some types of cationic photo initiators to serve as latent photochemical sources of very strong protonic or Lewis acids is generally the basis for their use in photo imaging applications. In some embodiments, a photoacid generator is an iodonium salt, a polonium salt, or a sulfonium salt. In some embodiments, a photoacid generator is (4-Methoxyphenyl)phenyliodonium or trifluoromethanesulfonate. In some embodiments, a photoacid generator is (2,4-dihydroxyphenyl)dimethylsulfonium triflate or (4 methoxyphenyl)dimethylsulfonium triflate, shown below:

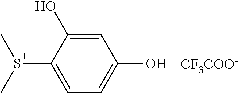

In some embodiments, a photoacid generator is iodonium and sulfonium salts of triflates, phosphates and/or antimonates. In some embodiments, a photoacid generator is about 0.5-5% by weight of the total formulation concentration. In some embodiments, a photoacid generator is about less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0 or greater than 7.0 weight % of the total formulation concentration.

In some embodiments, a photobase generator is 1,3-Bis[(2-nitrobenzyl)oxycarbonyl-4-piperidyl]propane or 1,3-Bis[(1-(9-fluorenylmethoxycarbonyl)-4-piperidyl]propane. The photobase generator should be present in a composition of the invention in an amount sufficient to enable deprotection of the monomer so that they are available for binding to the substrate. In some embodiments, a photobase generator is about 0.5-5% by weight of the total formulation concentration. In some embodiments, a photobase generator is about less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0 or greater than 7.0 weight % of the total formulation concentration.

In some embodiments, the photoresist formulation forms a photoresist layer on the surface of a microarray with the concentration of the photoresist layer being less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0 weight % of the total formulation concentration.

In some embodiments, the solvent is about 80-90 weight % of the total formulation concentration. In some embodiments, the solvent is about less than 70, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or greater than 99 weight % of the total formulation concentration.

In some embodiments, a polymer is a non-crosslinking inert polymer. In some embodiments, a polymer is a polyvinyl pyrrolidone. The general structure of polyvinyl pyrrolidone is as follows, where n is any positive integer greater than 1:

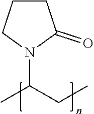

In some embodiments, a polymer is a polymer of vinyl pyrrolidone. In some embodiments, a polymer is polyvinyl pyrrolidone. Poly vinyl pyrrollidone is soluble in water and other polar solvents. When dry it is a light flaky powder, which generally readily absorbs up to 40% of its weight in atmospheric water. In solution, it has excellent wetting properties and readily forms films. In some embodiments, a polymer is a vinyl pyrrolidone or a vinyl alcohol. In some embodiments, a polymer is a polymethyl methacrylate.

In some embodiments, a polymer is 2.5-5% by weight of the total formulation concentration. In some embodiments, a polymer is about 0.5-5% by weight of the total formulation concentration. In some embodiments, a polymer is about less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0% by weight of the total formulation concentration.

In some embodiments, a solvent is water, ethyl lactate, n methyl pyrrollidone or a combination thereof. In some embodiments, ethyl lactate can be dissolved in water to more than 50% to form a solvent. In some embodiments, a solvent can be about 10% propylene glycol methyl ether acetate (PGMEA) and about 90% DI water. In some embodiments, a solvent can include up to about 20% PGMEA. In some embodiments, a solvent can include 50% ethyl lactate and 50% n methyl pyrrollidone. In some embodiments, a solvent is n methyl pyrrollidone. In some embodiments, a solvent is water, an organic solvent, or combination thereof. In some embodiments, the organic solvent is N Methyl pyrrolidone, di methyl formamide or combinations thereof.

In some embodiments, the solvent is about 80-90% by weight of the total formulation concentration. In some embodiments, the solvent is about less than 70, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or greater than 99% by weight of the total formulation concentration.

### Displacement Formulations

In some embodiments, a displacement formulation includes a mono-R₁ protected ethylenediamine that is 1-2% by weight of the total formulation concentration. In some embodiments, the mono-R₁ protected ethylenediamine is about 0.5-5% by weight of the total formulation concentration. In some embodiments, a mono-R₁ protected ethylenediamine is about less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0% by weight of the total formulation concentration. In some embodiments, the R₁ comprises a mono-protected amino group, e.g., a group protected via t-Boc or F-Moc chemistry. In most instances, increasing the concentration of the mono-R₁ protected ethylenediamine provides the best performance.

### Activating Formulations

Disclosed herein are activating formulations for activating carboxylic acid so that it reacts with a free amino group. In some embodiments, an activating formulation includes an activation agent (also referred to as a coupling reagent). In some embodiments, the coupling reagent is carbodiimide or triazole. In some embodiments, the carbodiimide is 1-ethyl-3-(3-dimethylaminopropyl) carbodiimide. In some embodiments, the carboxylic acid group activating compound is N-Hydroxysuccinimide (NHS). In some embodiments, the activating formulation optionally includes a solvent and/or a polymer.

In some embodiments, the activating formulation further includes an R₂-acetic acid, for example, bromoacetic acid, chloroacetic acid, fluoroacetic acid, iodoacetic acid. In some embodiments, the activating formulation further includes followed a coupling molecule, for example, PNA monomer acetic acid. In some embodiments, the PNA monomer acetic acid is R-thymine-1-acetic acid, R-(cytosine-1-yl)-acetic acid, R-adenine-9-yl-acetic acid, R-guanine-9-acetic acid, and R-uracil-1-acetic acid, where R is H or a protection group for the nucleic acid monomer and, in some embodiments, is Boc, Bis-Boc, Alloc, Benzoyl, Acetyl, Fmoc, Trityl, any of the above described amino protection group.

In some embodiments, the coupling reagent is selected from: 1-Ethyl-3-(3-dimethylaminopropyl)carbodiimide [EDC], N-hydroxysuccinimide [NHS], 1,3-Diisopropylcarbodiimide [DIC], hydroxybenzotriazole (HOBt), (O-(7-azabenzotriazol-1-yl)-N,N,N′,N′-tetramethyluronium hexafluorophosphate) [HATU], benzotriazol-1-yl-oxytripyrrolidinophosphonium hexafluorophosphate [PyBOP], and N,N-Diisopropylethylamine [DIEA]. In some embodiments, the solvent is water. In some embodiments, the solvent is N-methylpyrrolidone (NMP). In some embodiments, the coupling reagent converts the carboxylic acid to a carbonyl group (i.e., carboxylic acid group activation). In some embodiments, the carboxylic acid group is activated for 5, 10, 15, 20, 30, 45, or 60 minutes after exposure to a displacement reaction formulation.

In some embodiments, a coupling reagent is 2-4% by weight of the total formulation concentration. In some embodiments, a coupling reagent is about 0.5-5% by weight of the total formulation concentration. In some embodiments, a coupling reagent is about less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0% by weight of the total formulation concentration.

In any of the combinations above, the formulation can be completely water strippable. Thus, in some embodiments, water can be used to wash away the formulation after exposure.

In some embodiments, the activating formulation comprises 4% by weight of 1-ethyl-3-(3-dimethylaminopropyl) carbodiimide and 2% by weight of N-hydroxysuccinimide (NHS) dissolved in deionized water. In some embodiments, the activating formulation comprises 4% by weight of 1,3-Diisopropylcarbodiimide (DIC) and 2% by weight of hydroxybenzotriazole (HOBt) dissolved in NMP. In some embodiments, the activating formulation comprises 4% by weight of (O-(7-azabenzotriazol-1-yl)-N,N,N′,N′-tetramethyluronium hexafluorophosphate) (HATU) and 2% by weight of N,N-Diisopropylethylamine (DIEA) dissolved in NMP. In some embodiments, the activating formulation comprises 4% by weight of Benzotriazol-1-yl-oxytripyrrolidinophosphonium hexafluorophosphate (PyBOP) and 2% by weight of DIEA dissolved in NMP.

In some embodiments, the solvent is water. In some embodiments, the solvent is about 80-90% by weight of the total formulation concentration. In some embodiments, the solvent is about less than 70, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or greater than 99% by weight of the total formulation concentration.

In some embodiments, a polymer is a polyvinyl pyrrolidone and/or a polyvinyl alcohol. In some embodiments, a polymer is about 0.5-5% by weight of the total formulation concentration. In some embodiments, a polymer is about less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0% by weight of the total formulation concentration.

In some embodiments, a coupling reagent is a carbodiimide. In some embodiments, a coupling reagent is a triazole. In some embodiments, a coupling reagent is 1-ethyl-3-(3-dimethylaminopropyl) carbodiimide. In some embodiments, a coupling reagent is about 0.5-5% by weight of the total formulation concentration. In some embodiments, a coupling reagent is about less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0% by weight of the total formulation concentration.

### Linker Formulations

Also disclosed herein is a linker formulation. A linker formulation can include components such as a solvent, a polymer, a linker molecule, and a coupling reagent. In some embodiments, the polymer is 1% by weight polyvinyl alcohol and 2.5% by weight poly vinyl pyrrollidone, the linker molecule is 1.25% by weight polyethylene oxide, the coupling reagent is 1% by weight 1-ethyl-3-(3-dimethylaminopropyl) carbodiimide, and the solvent includes water. In some embodiments, the polymer is 0.5-5% by weight polyvinyl alcohol and 0.5-5% by weight poly vinyl pyrrollidone, the linker molecule is 0.5-5% by weight polyethylene oxide, the coupling reagent is 0.5-5% by weight 1-ethyl-3-(3-dimethylaminopropyl) carbodiimide, and the solvent includes water.

In some embodiments, the solvent is water, an organic solvent, or a combination thereof. In some embodiments, the organic solvent is N methyl pyrrolidone, dimethyl formamide, dichloromethane, dimethyl sulfoxide, or a combination thereof. In some embodiments, the solvent is about 80-90% by weight of the total formulation concentration. In some embodiments, the solvent is about less than 70, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or greater than 99% by weight of the total formulation concentration.

In some embodiments, a polymer is a polyvinyl pyrrolidone and/or a polyvinyl alcohol. The general structure of polyvinyl alcohol is as follows, where n is any positive integer greater than 1:

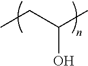

In some embodiments, a polymer is about 0.5-5% by weight of the total formulation concentration. In some embodiments, a polymer is about less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0% by weight of the total formulation concentration.

A linker molecule can be a molecule inserted between a surface disclosed herein and PNA chain that is being synthesized via a coupling molecule. A linker molecule does not necessarily convey functionality to the resulting PNA chain, such as molecular recognition functionality, but can instead elongate the distance between the surface and the PNA chain to enhance the exposure of the PNA chain's functionality region(s) on the surface. In some embodiments, a linker can be about 4 to about 40 atoms long to provide exposure. The linker molecules can be, for example, aryl acetylene, ethylene glycol oligomers containing 2-10 monomer units (PEGs), diamines, diacids, amino acids, and combinations thereof. Examples of diamines include ethylene diamine and diamino propane. Alternatively, linkers can be the same molecule type as that being synthesized (e.g., nascent polymers or various coupling molecules), such as polypeptides and polymers of amino acid derivatives such as for example, amino hexanoic acids, or PNA polymers. In some embodiments, a linker molecule is a molecule having a carboxylic group at a first end of the molecule and a protecting group at a second end of the molecule. In some embodiments, the protecting group is a t-Boc protecting group or an Fmoc protecting group. In some embodiments, a linker molecule is or includes an aryl acetylene, a polyethyleneglycol, a nascent polypeptide, a diamine, a diacid, a peptide, a PNA monomer or polymer, or combinations thereof. In some embodiments, a linker molecule is about 0.5-5% by weight of the total formulation concentration. In some embodiments, a linker molecule is about less than 0.1, 0.1, 0.2, 0.3, 0.4, 0.5, 0.6, 0.7, 0.8, 0.9, 1.0, 1.1, 1.2, 1.3., 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, or greater than 5.0% by weight of the total formulation concentration.

The unbound (or free end) portion of a linker molecule can have a reactive functional group which is blocked, protected, or otherwise made unavailable for reaction by a removable protecting group. The protecting group can be bound to a linker molecule to protect a reactive functionality on the linker molecule. Protecting groups that can be used include all acid- and base-labile protecting groups. For example, linker amine groups can be protected by t-butoxycarbonyl (t-BOC or BOC) or benzyloxycarbonyl (CBZ), both of which are acid labile, or by 9-fluorenylmethoxycarbonyl (FMOC), which is base labile.

Additional protecting groups that can be used include acid-labile groups for protecting amino moieties: tert-amyloxycarbonyl, adamantyloxycarbonyl, 1-methylcyclobutyloxycarbonyl, 2-(p-biphenyl)propyl(2)oxycarbonyl, 2-(p-phenylazophenylyl)propyl(2)oxycarbonyl, alpha,alpha-dimethyl-3,5-dimethyloxybenzyloxy-carbonyl, 2-phenylpropyl(2)oxycarbonyl, 4-methyloxybenzyloxycarbonyl, furfuryloxycarbonyl, triphenylmethyl (trityl), p-toluenesulfenylaminocarbonyl, dimethylphosphinothioyl, diphenylphosphinothioyl, 2-benzoyl-1-methylvinyl, o-nitrophenylsulfenyl, and 1-naphthylidene; as base labile groups for protecting amino moieties: 9 fluorenylmethyloxycarbonyl, methylsulfonylethyloxycarbonyl, and 5-benzisoazolylmethyleneoxycarbonyl; as groups for protecting amino moieties that are labile when reduced: dithiasuccinoyl, p-toluene sulfonyl, and piperidino-oxycarbonyl; as groups for protecting amino moieties that are labile when oxidized: (ethylthio)carbonyl; as groups for protecting amino moieties that are labile to miscellaneous reagents, the appropriate agent is listed in parenthesis after the group: phthaloyl (hydrazine), trifluoroacetyl (piperidine), and chloroacetyl (2-aminothiophenol); acid-labile groups for protecting carboxylic acids: tert-butyl ester; acid labile groups for protecting hydroxyl groups: dimethyltrityl. See also, Greene, T. W., Protective Groups in Organic Synthesis, Wiley-Interscience, NY, (1981).

In any of the combinations above, the formulations can be completely water strippable.

### Substrates

Also disclosed herein are substrates. In some embodiments, a substrate surface is planar (i.e., 2-dimensional). In some embodiments, a substrate surface is functionalized with free amine groups. In some embodiments, a substrate surface is functionalized with free carboxylic acid groups. A surface that is functionalized with free amine groups can be converted to free carboxylic acid groups by reacting with activating the carboxylic acid groups of a molecule comprising at least two free carboxylic acid groups (e.g., converting the carboxylic acid group to a carbonyl group using carbodiimide) and reacting the molecule with the free amine groups attached to the surface of the substrate. In some embodiments, the molecule comprising multiple carboxylic acid groups is succinic anhydride, polyethylene glycol diacid, benzene-1,3,5-tricarboxylic acid, benzenehexacarboxylic acid, or carboxymethyl dextran.

In some aspects, a surface is a material or group of materials having rigidity or semi-rigidity. In some aspects, a surface can be substantially flat, although in some aspects it can be desirable to physically separate synthesis regions for different molecules or features with, for example, wells, raised regions, pins, pillars, etched trenches, or the like. In certain aspects, a surface may be porous. Surface materials can include, for example, silicon, bio-compatible polymers such as, for example poly(methyl methacrylate) (PMMA) and polydimethylsiloxane (PDMS), glass, SiO₂ (such as, for example, a thermal oxide silicon wafer such as that used by the semiconductor industry), quartz, silicon nitride, functionalized glass, gold, platinum, and aluminum. Functionalized surfaces include for example, amino-functionalized glass, carboxy functionalized glass, and hydroxy functionalized glass. Additionally, a surface may optionally be coated with one or more layers to provide a second surface for molecular attachment or functionalization, increased or decreased reactivity, binding detection, or other specialized application. Surface materials and or layer(s) can be porous or non-porous. For example, a surface can be comprised of porous silicon. Additionally, the surface can be a silicon wafer or chip such as those used in the semiconductor device fabrication industry. In the case of a wafer or chip, a plurality of arrays can be synthesized on the wafer.

In some embodiments, a substrate can include a porous layer (i.e., a 3-dimensional layer) comprising functional groups for binding a first monomer building block. In some embodiments, a substrate surface comprises pillars for PNA attachment or synthesis. In some embodiments, a porous layer is added to the top of the pillars.

### Porous Layer Substrates

Porous layers that can be used are flat, permeable, polymeric materials of porous structure that have an amine group (that is native to the constituent polymer or that is introduced to the porous layer) for attachment of the first PNA building block. For example, a porous layer can be comprised of porous silicon with functional groups for attachment of a polymer building block attached to the surface of the porous silicon. In another example, a porous layer can comprise a cross-linked polymeric material. In some embodiments, the porous layer can employ polystyrenes, saccharose, dextrans, polyacryloylmorpholine, polyacrylates, polymethylacrylates, polyacrylamides, polyacrylolpyrrolidone, polyvinylacetates, polyethyleneglycol, agaroses, sepharose, other conventional chromatography type materials and derivatives and mixtures thereof. In some embodiments, the porous layer building material is selected from: poly(vinyl alcohol), dextran, sodium alginate, poly(aspartic acid), poly(ethylene glycol), poly(ethylene oxide), poly(vinyl pyrrolidone), poly(acrylic acid), poly(acrylic acid)-sodium salt, poly(acrylamide), poly(N-isopropyl acrylamide), poly(hydroxyethyl acrylate), poly(acrylic acid), poly(sodium styrene sulfonate), poly(2-acrylamido-2-methyl-1-propanesulfonic acid), polysaccharides, and cellulose derivatives. Preferably the porous layer has a porosity of 10-80%. In one embodiment, the thickness of the porous layer ranges from 0.01 μm to about 1,000 μm. Pore sizes included in the porous layer may range from 2 nm to about 100 μm.

According to another embodiment of the present invention there is provided a substrate comprising a porous polymeric material having a porosity from 10-80%, wherein reactive groups are chemically bound to the pore surfaces and are adapted in use to interact, e.g. by binding chemically, with a reactive species, e.g., deprotected monomeric building blocks or polymeric chains. In some embodiments, the reactive group is a free amine group. The free amine group is free to bind, for example, an activated carboxylic group of a coupling molecule or substituted acetic acid molecule.

In an embodiment, the porous layer is in contact with a support layer. The support layer comprises, for example, metal, plastic, silicon, silicon oxide, or silicon nitride. In another embodiment, the porous layer can be in contact with a patterned surface, such as on top of pillar substrates described below.

### Pillar Substrates

In some embodiments, a substrate can include a planar layer having an upper surface and a lower surface; and a plurality of pillars operatively coupled to the layer in positionally-defined locations, wherein each pillar has a planar surface extended from the layer, wherein the distance between the surface of each pillar and the upper surface of the layer is between about 1,000-5,000 angstroms, and wherein the plurality of pillars are present at a density of greater than about 10,000/cm².

In some embodiments, the planar layer comprises metal, plastic, silicon, silicon oxide, or silicon nitride. In some embodiments, the metal is chromium. In some embodiments, the metal is chromium, titanium, aluminum, tungsten, gold, silver, tin, lead, thallium, indium, or a combination thereof. In some embodiments, the planar layer is at least 98.5-99% (by weight) metal, plastic, silicon, silicon oxide, or silicon nitride. In some embodiments, the planar layer is 100% metal, silicon, silicon oxide, or silicon nitride. In some embodiments, the planar layer is at least about greater than 90, 91, 92, 93, 94, 95, 96, 97, 98, 98.5, or 99% metal, silicon, silicon oxide, or silicon nitride. In some embodiments, the layer is a homogenous layer of metal, silicon, silicon oxide, or silicon nitride.

In some embodiments, the distance between the surface of each pillar and the upper surface of the planar layer can be between about less than 1,000, 2,000, 3,000, 3,500, 4,500, 5,000, or greater than 5,000 angstroms (or any integer in between).

In some embodiments, the surface of each pillar is parallel to the upper surface of the layer. In some embodiments, the surface of each pillar is substantially parallel to the upper surface of the layer.

In some embodiments, the plurality of pillars are present at a density of greater than 500, 1,000, 2,000, 3,000, 4,000, 5,000, 6,000, 7,000, 8,000, 9,000, 10,000, 11,000, or 12,000/cm² (or any integer in between). In some embodiments, the plurality of pillars are present at a density of greater than 10,000/cm². In some embodiments, the plurality of pillars are present at a density of about 10,000/cm² to about 2.5 million/cm² (or any integer in between). In some embodiments, the plurality of pillars are present at a density of greater than 2.5 million/cm².

In some embodiments, the surface area of each pillar surface is at least 1 μm². In some embodiments, the surface area of each pillar surface can be at least 0.1, 0.5, 12, 3, 4, 5, 6, 7, 8, 9, 10, 15, 20, 25, 30, 35, 40, 45, or 50 μm² (or any integer in between). In some embodiments, the surface area of each pillar surface has a total area of less than 10,000 μm². In some embodiments, the surface area of each pillar surface has a total area of less than 500, 1,000, 2,000, 3,000, 4,000, 5,000, 6,000, 7,000, 8,000, 9,000, 10,000, 11,000, or 12,000 μm² (or any integer in between).

In some embodiments, the distance between the surface of each pillar and the lower surface of the layer is 2,000-7,000 angstroms. In some embodiments, the distance between the surface of each pillar and the lower surface of the layer is about less than 500, 1,000, 2,000, 3,000, 4,000, 5,000, 6,000, 7,000, 8,000, 9,000, 10,000, 11,000, 12,000, or greater than 12,000 angstroms (or any integer in between). In some embodiments, the distance between the surface of each pillar and the lower surface of the layer is 7,000, 3,000, 4,000, 5,000, 6,000, or 7,000 angstroms (or any integer in between).

In some embodiments, the layer is 1,000-2,000 angstroms thick. In some embodiments, the layer is about less than 500, 1,000, 2,000, 3,000, 4,000, 5,000, 6,000, 7,000, 8,000, 9,000, 10,000, 11,000, 12,000, or greater than 12,000 angstroms thick (or any integer in between).

In some embodiments, the center of each pillar is at least 2,000 angstroms from the center of any other pillar. In some embodiments, the center of each pillar is at least about 500, 1,000, 2,000, 3,000, or 4,000 angstroms (or any integer in between) from the center of any other pillar. In some embodiments, the center of each pillar is at least about 2 μm to 200 μm from the center of any other pillar.

In some embodiments, at least one or each pillar comprises silicon. In some embodiments, at least one or each pillar comprises silicon dioxide or silicon nitride. In some embodiments, at least one or each pillar is at least 90, 91, 92, 93, 94, 95, 96, 97, 98, 98.5, or 99% (by weight) silicon dioxide.

In some embodiments, a substrate can include a linker molecule having a free amino terminus attached to the surface of each pillar. In some embodiments, a substrate can include a linker molecule having a free amino terminus attached to the surface of at least one pillar. In some embodiments, a substrate can include a linker molecule having a protecting group attached to the surface of each pillar. In some embodiments, a substrate can include a linker molecule having a protecting group attached to the surface of at least one pillar. In some embodiments, a substrate can include a coupling molecule attached to the surface of at least one pillar. In some embodiments, a substrate can include a coupling molecule attached to the surface of each pillar. In some embodiments, a substrate can include a polymer in contact with the surface of at least one of the pillars. In some embodiments, a substrate can include a polymer in contact with the surface of each pillar. In some embodiments, a substrate can include a gelatinous form of a polymer in contact with the surface of at least one of the pillars. In some embodiments, a substrate can include a solid form of a polymer in contact with the surface of at least one of the pillars.

In some embodiments, the surface of at least one of the pillars of the substrate is derivatized. In some embodiments, a substrate can include a polymer chain attached to the surface of at least one of the pillars. In some embodiments, the polymer chain comprises a PNA chain. In some embodiments, the attachment to the surface of the at least one pillar is via a covalent bond.

In some embodiments, the surface of each pillar is square or rectangular in shape. In some embodiments, the substrate can be coupled to a silicon dioxide layer. The silicon dioxide layer can be about 0.5 μm to 3 μm thick. In some embodiments, the substrate can be coupled to a wafer, e.g., a silicon wafer. The silicon dioxide layer can be about 700 μm to 750 μm thick.

### Arrays

Also disclosed herein are arrays. In some embodiments, an array can be a two-dimensional array. In some embodiments, the array comprises a surface comprising a substrate and the substrate comprising: a planar layer having an upper surface and a lower surface.

In some embodiments, a two-dimensional array can include features attached to a surface at positionally-defined locations, said features each comprising: a collection of PNA chains or PNA-DNA chimeric oligonucleotide chains of determinable sequence and intended length, wherein within an individual feature, the fraction of PNA or PNA-DNA chimeric oligonucleotides within said collection having the intended length is characterized by an average coupling efficiency for each coupling step of about 98%. In some embodiments, the array comprises a plurality of pillars operatively coupled to the layer in the positionally-defined locations, wherein each pillar has a planar surface extended from the layer, wherein the distance between the surface of each pillar and the upper surface of the layer is between 1,000-5,000 angstroms, and wherein the plurality of pillars are present at a density of greater than 10,000/cm².

In some embodiments, the surface of the array is functionalized with free amine groups. In some embodiments, the surface density of free amine groups on the array is greater than 10/cm², 100/cm², 1,000/cm², 10,000/cm², 100,000/cm², 1,000,000/cm², or 10,000,000/cm².

In some embodiments, the surface density of the features on the array is greater than 10/cm², 100/cm², 1,000/cm², 10,000/cm², 100,000/cm², 1,000,000/cm², or 10,000,000/cm².

In some embodiments, an array can be a three-dimensional array, e.g., a porous array comprising features attached to the surface of the porous array. In some embodiments, the surface of a porous array includes external surfaces and surfaces defining pore volume within the porous array. In some embodiments, a three-dimensional array can include features attached to a surface at positionally-defined locations, said features each comprising: a collection of PNA chains or PNA-DNA chimeric oligonucleotide chains of determinable sequence and intended length. In one embodiment, within an individual feature, the fraction of PNA chains or PNA-DNA chimeric oligonucleotide chains within said collection having the intended length is characterized by an average coupling efficiency for each coupling step of greater than 98%.

In some embodiments, the average coupling efficiency for each coupling step for PNA-PNA binding is at least 98.5%. In some embodiments, the average coupling efficiency for each coupling step for PNA-PNA binding is at least 99%. In some embodiments, the average coupling efficiency for each coupling step for PNA-PNA binding is at least 90, 91, 92, 93, 94, 95, 96, 97, 98, 98.5, 98.6,98.7, 98.8, 98.9, 99.0, 99.1, 99.2, 99.3, 99.4, 99.5, 99.6, 99.7, 99.8, 99.9, or 100%.

In some embodiments, the average coupling efficiency for each coupling step for PNA-DNA binding is at least 97%. In some embodiments, the average coupling efficiency for each coupling step for PNA-DNA binding is at least 90, 91, 92, 93, 94, 95, 96, 97, 98, 98.5, 98.6,98.7, 98.8, 98.9, 99.0, 99.1, 99.2, 99.3, 99.4, 99.5, 99.6, 99.7, 99.8, 99.9, or 100%.

In some embodiments, the average coupling efficiency for each coupling step for DNA-DNA binding is at least 98.5%. In some embodiments, the average coupling efficiency for each coupling step for DNA-DNA binding is at least 90, 91, 92, 93, 94, 95, 96, 97, 98, 98.5, 98.6,98.7, 98.8, 98.9, 99.0, 99.1, 99.2, 99.3, 99.4, 99.5, 99.6, 99.7, 99.8, 99.9, or 100%.

In some embodiments, the average coupling efficiency for each coupling step for a full length PNA or PNA-DNA chimeric oligonucleotide synthesis is at least 98.5%. In some embodiments, the average coupling efficiency for each coupling step is at least 99%. In some embodiments, the average coupling efficiency for each coupling step is at least 90, 91, 92, 93, 94, 95, 96, 97, 98, 98.5, 98.6,98.7, 98.8, 98.9, 99.0, 99.1, 99.2, 99.3, 99.4, 99.5, 99.6, 99.7, 99.8, 99.9, or 100%.

In some embodiments, the purity of each feature with regards to the fraction of full-length predetermined PNA chains or PNA-DNA chimeric oligonucleotide chains is a fraction F of the full-length predetermined PNA chains or PNA-DNA chimeric oligonucleotide chains of each feature having a predetermined sequence and a predetermined full-length sequence length N being characterized by F=10^{(N+1)·log (E/100%)}. In some embodiments, F is characterized by an average coupling efficiency E of at least 98.5% for coupling each monomer of the predetermined sequence. In some embodiments, F is characterized by an average coupling efficiency E of at least 98.5% for coupling each monomer of the predetermined sequence. In some embodiments, the average coupling efficiency E for each coupling step is 90, 91, 92, 93, 94, 95, 96, 97, 98, 98.5, 98.6,98.7, 98.8, 98.9, 99.0, 99.1, 99.2, 99.3, 99.4, 99.5, 99.6, 99.7, 99.8, 99.9, or 100%.

In some embodiments, the distribution of sequence lengths on the array based on the synthesis of a PNA or PNA-DNA chimeric sequence of a defined length (e.g., a 64 mer). At each coupling step, the length of a sequence where a desired coupling does not occur becomes fixed at that length when a capping solution is used. The distribution of lengths according to the step yield for each sequence length less than the full sequence is given by the following equation:

F(N)=10^{(N+1)·log (E/100%)}−10^{(N)·log (E/100%)}

where F(N) is the proportion of sequences on the array at a length N that are less than the full length sequence, and where E is the average coupling efficiency percentage. The precise value of E at each length N can also be used to generate an exact number of oligomers at each length.

The proportion of full length sequence is given by the following equation:

F(N)=10^{(N)·log (E/100%)}

where F(N) is the proportion of sequences on the array of a full length sequence (no further coupling steps), and where E is the average coupling efficiency.

In some embodiments, the sequence length N is at least 64 monomers in length and the fraction of the less than full-length predetermined PNA chains or PNA-DNA chimeric oligonucleotide chains equaling (1−F). In some embodiments, the sequence length N is at least 65 monomers in length.

In some embodiments, each PNA chain or PNA-DNA chimeric oligonucleotide chain is from 5 to 100 monomers in length. In some embodiments, each PNA chain or PNA-DNA chimeric oligonucleotide chain is at least 64 monomers in length. In some embodiments, each PNA chain or PNA-DNA chimeric oligonucleotide chain is at least 65 monomers in length. In some embodiments, each PNA chain or PNA-DNA chimeric oligonucleotide chain is at least 100 monomers or greater than 100 monomers in length. In some embodiments, each PNA chain or PNA-DNA chimeric oligonucleotide chain is at least 5, 10, 15, 20, 25, 30, 35, 40, 45, 50, 55, 60, 65, 70, 75, 80, 85, 90, 95, or 100 monomers in length. In some embodiments, each PNA chain or PNA-DNA chimeric oligonucleotide chain is less than 5, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or greater than 100 monomers in length. In some embodiments, each PNA chain or PNA-DNA chimeric oligonucleotide chain comprises one or more L-chiral PNA monomers. In some embodiments, each PNA chain or PNA-DNA chimeric oligonucleotide chain comprises one or more D-chiral PNA monomers. In some embodiments, each PNA chain or PNA-DNA chimeric oligonucleotide chain comprises one or more modified nucleotides.

In some embodiments, each PNA-DNA oligonucleotide chimeric chain is from 5 to 100 monomers in length. In some embodiments, each PNA-DNA oligonucleotide chimeric chain is at least 64 monomers in length. In some embodiments, each PNA-DNA oligonucleotide chimeric chain is at least 65 monomers in length. In some embodiments, each PNA-DNA oligonucleotide chimeric chain is at least 100 monomers or greater than 100 monomers in length. In some embodiments, each PNA-DNA oligonucleotide chimeric chain is at least 5, 10, 15, 20, 25, 30, 35, 40, 45, 50, 55, 60, 65, 70, 75, 80, 85, 90, 95, or 100 monomers in length. In some embodiments, each PNA-DNA oligonucleotide chimeric chain is less than 5, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or greater than 100 monomers in length. In some embodiments, each PNA-DNA oligonucleotide chimeric chain comprises one or more L-chiral PNA monomers. In some embodiments, each PNA-DNA oligonucleotide chimeric chain comprises one or more D-chiral PNA monomers. In some embodiments, each PNA-DNA oligonucleotide chimeric chain comprises one or more modified nucleotides. In some embodiments, each PNA-DNA oligonucleotide chimeric chain comprises one or more ribonucleotides at a DNA position.

In some embodiments, an array can include at least 1,000 different features attached to the surface. In some embodiments, an array can include at least 10,000 different features attached to the surface. In some embodiments, an array can include at least 100, 500, 1000, 2000, 3000, 4000, 5000, 6000, 7000, 8000, 9000, 10,000, or greater than 10,000 different features attached to the surface (or any integer in between).

In some embodiments, an array can include at least 1,000 different PNA chains or PNA-DNA chimeric oligonucleotide chains attached to the surface. In some embodiments, an array can include at least 10,000 different PNA chains or PNA-DNA chimeric oligonucleotide chains attached to the surface. In some embodiments, an array can include at least 100, 500, 1000, 2000, 3000, 4000, 5000, 6000, 7000, 8000, 9000, 10,000, or greater than 10,000 different PNA chains or PNA-DNA chimeric oligonucleotide chains attached to the surface (or any integer in between).

In some embodiments, each feature comprises at least 500 identical full-length PNA chains or PNA-DNA chimeric oligonucleotide chains, wherein each identical full-length PNA chain or PNA-DNA chimeric oligonucleotide chain has a predetermined full-length of at least 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or greater than 100 monomers in length. In some embodiments, each feature comprises 100, 200, 300, 400, 500, 600, 700, 800, 900, 1000, 1100, 1200, 1300, 1400, 1500, 1600, 1700, 1800, 1900, or 2000 identical full-length PNA chains or PNA-DNA chimeric oligonucleotide chains, wherein each identical full-length PNA chain or PNA-DNA chimeric oligonucleotide chain has a predetermined full-length of at least 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or greater than 100 monomers in length.

In some embodiments, each of the positionally-defined locations is at a different, known location that is physically separated from each of the other positionally-defined locations. In some embodiments, each of the positionally-defined locations is a positionally-distinguishable location. In some embodiments, each determinable sequence is a known sequence. In some embodiments, each determinable sequence is a distinct sequence.

In some embodiments, each feature is attached to a surface of the array at a different positionally-defined location and the positionally-defined location of each feature corresponds to a positionally-defined location of a pillar, wherein the top surface of each pillar is at least 1 μm² in size.

In some embodiments, the features are covalently attached to the surface. In some embodiments, said PNA chains or PNA-DNA chimeric oligonucleotide chains are attached to the surface through a linker molecule or a coupling molecule.

In some embodiments, the features comprise a plurality of distinct, nested, overlapping PNA chains or PNA-DNA chimeric oligonucleotide chains comprising subsequences derived from a source DNA or RNA sequence having a known sequence. In some embodiments, each PNA chain or PNA-DNA chimeric oligonucleotide chain in the plurality is substantially the same length. In some embodiments, each PNA chain or PNA-DNA chimeric oligonucleotide chain in the plurality is the same length.

In some embodiments, each PNA chain or PNA-DNA chimeric oligonucleotide chains in the plurality is at least 5 monomers in length. In some embodiments, each PNA chain or PNA-DNA chimeric oligonucleotide chain in the plurality is at least 5, 10, 15, 20, 25, 30, 35, 40, 45, 50, 55, or 60 monomers in length. In some embodiments, each PNA chain or PNA-DNA chimeric oligonucleotide chain in the plurality is less than 5, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or greater than 100 monomers in length. In some embodiments, each PNA chain or PNA-DNA chimeric oligonucleotide chain in the plurality is less than 5, at least 100, 105, 110, 115, 120, 125, 130, 135, 140, 145, 150, 155, 160, 165, 170, 175, 180, 185, 190, 195, 200 or greater than 200 monomers in length

In some embodiments, at least one PNA chain or PNA-DNA chimeric oligonucleotide chain in the plurality is at least 5 monomers in length. In some embodiments, at least one PNA chain or PNA-DNA chimeric oligonucleotide chains in the plurality is at least 5, 10, 15, 20, 25, 30, 35, 40, 45, 50, 55, or 60 monomers in length. In some embodiments, at least one PNA chain or PNA-DNA chimeric oligonucleotide chain in the plurality is less than 5, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or greater than 100 monomers in length.

In some embodiments, each PNA chain or PNA-DNA chimeric oligonucleotide chain in a feature is substantially the same length. In some embodiments, each PNA chain or PNA-DNA chimeric oligonucleotide chain in a feature is the same length. In some embodiments, the features comprise a plurality of PNA chains or PNA-DNA chimeric oligonucleotide chains each having a random, determinable sequence of monomers.

### Methods

### Methods of Manufacturing Substrates

Also disclosed herein are methods for making substrates. In some embodiments, a method of producing a substrate can include coupling a porous layer to a support layer. The support layer can comprise any metal or plastic or silicon or silicon oxide or silicon nitride. In one embodiment, the substrate comprises multiple amino group substrates attached to the substrate for binding PNA monomers during PNA or PNA-DNA chimeric oligonucleotide synthesis and PNA monomer coupling. In some embodiments, a method of producing a substrate can include coupling a porous layer to a plurality of pillars, wherein the porous layer comprises functional groups for attachment of a compound to the substrate, wherein the plurality of pillars are coupled to a planar layer in positionally-defined locations, wherein each pillar has a planar surface extended from the planar layer, wherein the distance between the surface of each pillar and the upper surface of the planar layer is between about 1,000-5,000 angstroms, and wherein the plurality of pillars are present at a density of greater than about 10,000/cm².

In some embodiments, the surface of each pillar is parallel to the upper surface of the planar layer. In some embodiments, the surface of each pillar is substantially parallel to the upper surface of the planar layer.

### Surface Derivatization

Substrates can be surface derivatized in a semiconductor module as explained in U.S. Patent Publication No. 2010/0240555 (filed on Sep. 29, 2006), herein incorporated by reference, in its entirety, for all purposes. A typical substrate of the present invention has pillars of oxide ready to be surface derivatized. Surface derivatization is a method wherein an amino silane group is added to the substrate so that free amino groups are available for coupling the biomolecules. In some aspects, the first molecule to be attached to the surface derivatized substrate is a tboc protected Glycine. This coupling procedure is similar to a standard Merrifield solid phase peptide synthesis procedure, which is generally known to one skilled in this art.

In some embodiments, a method of preparing a substrate surface can include obtaining a surface comprising silicon dioxide and contacting the surface with a photoresist formulation comprising a photoactive compound and a photo-protective compound, optionally including a polymer and a solvent; and applying ultraviolet light to positionally-defined locations located on the top of the surface and in contact with the photoresist formulation. In some aspects, the method can include removing the photoresist formulation located external to the positionally-defined locations.

### Methods of Manufacturing Arrays

Also disclosed herein are methods for manufacturing arrays. In some embodiments, the arrays disclosed herein can be synthesized in situ on a surface, e.g., a substrate disclosed herein. In some instances, the arrays are made using photolithography. For example, the substrate is contacted with a photoresist formulation. Masks can be used to control radiation or light exposure to specific locations on a surface provided with free linker molecules or free coupling molecules having protecting groups. In the exposed locations, the protecting groups are removed, resulting in one or more newly exposed reactive moieties on the coupling molecule or linker molecule. The desired linker or coupling molecule is then coupled to the unprotected attached molecules, e.g., at the free amine group. The process can be repeated to synthesize a large number of features in specific or positionally-defined locations on a surface (see, for example, U.S. Pat. No. 5,143,854 to Pirrung et al., U.S. Patent Publication Nos. 2007/0154946 (filed on Dec. 29, 2005), 2007/0122841 (filed on Nov. 30, 2005), 2007/0122842 (filed on Mar. 30, 2006), 2008/0108149 (filed on Oct. 23, 2006), and 2010/0093554 (filed on Jun. 2, 2008), each of which is herein incorporated by reference).

In some embodiments, a method of producing a three-dimensional (e.g., porous) array of features, can include obtaining a porous layer attached to a surface; and attaching the features to the porous layer, said features each comprising a collection of PNA chains or PNA-DNA chimeric oligonucleotide chains of determinable sequence and intended length, wherein within an individual feature, the fraction of PNA chains or PNA-DNA chimeric oligonucleotide chains within said collection having the intended length is characterized by an average coupling efficiency for each coupling step of at least about 98%. In some embodiments, the features are attached to the surface using a photoresist formulation, comprising a photoactive compound, a photo-protective compound, and optionally: a polymer, and a solvent. In some embodiments, the features are attached to the surface using a photoresist formulation disclosed herein. In some embodiments, the photoresist formulation is stripped away using water.

In some embodiments, described herein is a process of manufacturing an array. A surface comprising attached free amine groups is provided. The surface is contacted with a photoresist formulation comprising a photoactive compound, a photo-protective compound, and optionally: a polymer, and a solvent. The surface is exposed to electromagnetic radiation, for example, ultraviolet (UV) light in a deep ultra violet scanner tool according to a pattern defined by a photomask, wherein the locations exposed to radiation undergo photoacid generation due to the presence of a photoacid generator in the photoresist formulation. The expose energy can be from 1 mJ/cm² to 100 mJ/cm² in order to produce enough photobase. In some embodiments, the radiation includes UV light at 248 nm. In some embodiments, the radiation includes 365 nm ultraviolet light. Radiation to activate a photoacid or photobase generator can be in a range of wavelengths, and is not limited to wavelengths disclosed herein.

The photo-protective compound shields any molecules attached to the microarray from the electromagnetic radiation, preventing the possible radiation-induced destruction of the attached molecules. Photo-protective compounds include, but are not limited to titanium dioxide, zinc sulfide, magnesium fluoride, and the like

The surface is post baked upon exposure in a post exposure bake module. Post exposure bake acts as a chemical amplification step. The baking step amplifies the initially generated photoacid and also enhances the rate of diffusion to the substrate. The post bake temperature can vary between 75° Celsius to 115° Celsius, depending on the thickness of the porous surface or the planar layer of the substrate, for at least 60 seconds and not usually exceeding 120 seconds. The free amine group is coupled to an activated carboxylic acid group of a free compound molecule, resulting in coupling of the free compound molecule to the amine group attached to the surface. This surface may be a porous surface or the planar layer of the substrate. The synthesis of PNA chains or PNA-DNA chimeric oligonucleotide chains coupled to an amine group attached to the surface occurs in a C→N synthesis orientation. Alternatively, a diamine linker may be attached to a free carboxylic acid group to orient synthesis in a C→N direction, with the activated carboxylic acid group of free compound molecule attaching to amine groups bound to the surface of the substrate.

The photoresist formulation can now be stripped away. In some embodiments, provided herein is a method of stripping the photoresist completely with deionized (DI) water. This process is accomplished in a developer module. The wafer is spun on a vacuum chuck for, e.g., 60 seconds to 90 seconds and deionized water is dispensed through a nozzle for about 30 seconds.

The photoresist formulation can be applied to the surface in a coupling spin module. A coupling spin module can typically have 20 nozzles or more to feed the photoactive coupling formulation. These nozzles can be made to dispense the photoactive coupling formulation by means of pressurizing the cylinders that hold these solutions or by a pump that dispenses the required amount. In some embodiments, the pump is employed to dispense 5-8 cc of the photoactive coupling formulation onto the substrate. The substrate is spun on a vacuum chuck for 15-30 seconds and the photoactive coupling formulation is dispensed. The spin speed can be set to 2000 to 2500 rpm.

Subsequent steps of the method are described above, and are illustrated in FIG. 1, Scheme 1, and FIG. 2A and 2B, as described earlier under “Compositions.”

Optionally, a cap film solution coat is applied on the surface to prevent the unreacted amino groups on the substrate from reacting with the next coupling molecule. The cap film coat solution can be prepared as follows: a solvent, a polymer, and a coupling molecule. The solvent that can be used can be an organic solvent like N methyl pyrrolidone, dimethyl formamide, or combinations thereof. The capping molecule is typically acetic anhydride and the polymer can be polyvinyl pyrrolidone, polyvinyl alcohol, polymethyl methacrylate, poly (methyl iso propenyl) ketone, or poly (2 methyl pentene 1 sulfone). In some embodiments, the capping molecule is ethanolamine.

This process is done in a capping spin module. A capping spin module can include one nozzle that can be made to dispense the cap film coat solution onto the substrate. This solution can be dispensed through pressurizing the cylinder that stores the cap film coat solution or through a pump that precisely dispenses the required amount. In some embodiments, a pump is used to dispense around 5-8 cc of the cap coat solution onto the substrate. The substrate is spun on a vacuum chuck for 15-30 s and the coupling formulation is dispensed. The spin speed can be set to 2000 to 2500 rpm.

The substrates with the capping solution are baked in a cap bake module. A capping bake module is a hot plate set up specifically to receive wafers just after the capping film coat is applied. In some embodiments, provided herein is a method of baking the spin coated capping coat solution in a hot plate to accelerate the capping reaction significantly. Hot plate baking generally reduces the capping time to less than two minutes.

The byproducts of the capping reaction are stripped in a stripper module. A stripper module can include several nozzles, typically up to 10, set up to dispense organic solvents such as acetone, iso propyl alcohol, N methyl pyrrolidone, dimethyl formamide, DI water, etc. In some embodiments, the nozzles can be designated for acetone followed by iso propyl alcohol to be dispensed onto the spinning wafer. The spin speed is set to be 2000 to 2500 rpm for around 20 seconds.

This entire cycle can be repeated as desired with different coupling molecules each time to obtain a PNA chain or PNA-DNA chimeric oligonucleotide chain of determinable sequence and intended length.

### Methods of Use of PNA or PNA-DNA Microarrays

Also disclosed herein are methods of using substrates, formulations, and/or arrays. Uses of the arrays disclosed herein can include research applications, therapeutic purposes, medical diagnostics, and/or stratifying one or more patients.

Any of the arrays described herein can be used as a research tool or in a research application. In one aspect, arrays can be used for high throughput screening assays. For example, PNA substrates can be tested by subjecting the array to a DNA or RNA molecule and identifying the presence or absence of the complimentary DNA, RNA, or PNA molecule, e.g., by detecting at least one change among the features of the array. PNA-DNA chimeric substrates can be tested by subjecting the array to a complementary DNA molecule and performing a single nucleotide extension reaction to determine whether the substrate is biologically active, and to identify a SNP in a sample.

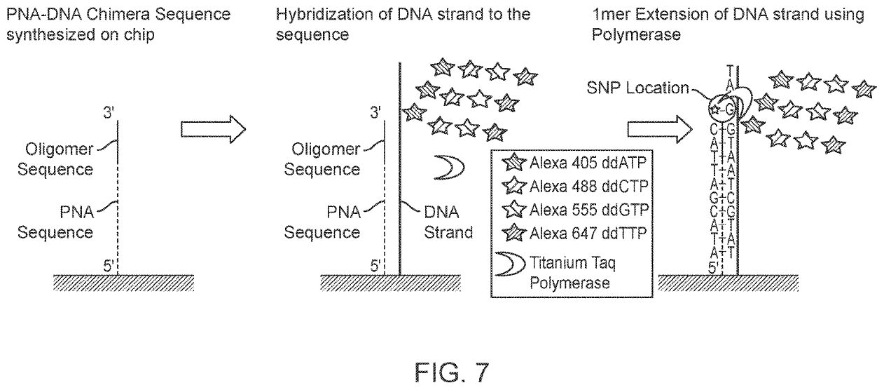

In some embodiments, an array can be used for detection of sequence variants in a sample, e.g., single nucleotide polymorphisms (SNPs). Detection of sequence variants can occur through observing sequence-specific hybridization of labeled molecules to a probe on an array. Detection of sequence variants can also occur through binding of a sequence suspected of having a sequence variant to a probe on an array, followed by performing a polymerase extension reaction with a labelled nucleotides. In preferred embodiments, PNA-DNA chimeric oligonucleotide probes are bound to the array and hybridize to nucleotide sequences from a sample suspected of comprising a sequence variant. The PNA-DNA chimeric oligonucleotides are enzymatically active, i.e., they are capable of acting as a substrate for complementary nucleotide incorporation into a growing strand using a polymerase under preferred conditions for polymerization. FIG. 7 provides an exemplary scheme for detecting the identity of a sample oligonucleotide hybridized to a PNA-DNA chimeric oligonucleotide covalently attached to the array using a polymerase-based single nucleotide extension reaction with a labeled nucleotide. Examples of PNA-DNA chimeric oligonucleotide-based methods for SNP detection are provided in U.S. Pat. No. 6,316,230, incorporated herein by reference in its entirety.

Arrays can also be used in screening assays for ligand binding, to determine substrate specificity, or for the identification of complimentary DNA, RNA, PNA molecule that are expressed in certain cells in vivo or in vitro. Labeling techniques, protease assays, as well as binding assays useful for carrying out these methodologies are generally well-known to one of skill in the art.

In some embodiments, an array can be used to represent a predefined PNA chain as a sequence of overlapping PNA sequences. For example, the PNA sequence of a known gene is divided into overlapping sequence segments of any length and of any suitable overlapping frame, and PNA chains corresponding to the respective sequence segments are in-situ synthesized as disclosed herein. The individual PNA segments so synthesized can be arranged starting from the amino terminus of the predefined PNA chain.

In some embodiments, a sample is applied to an array having a plurality of random PNA chains. The random PNA chains can be screened and BLASTed to determine homologous domains with, e.g., a 90% or more identity to a given nucleotide sequence. In some aspect, the whole PNA sequence can then be synthesized and used to identify potential markers and/or causes of a disease of interest.

In some embodiments, an array is used for high throughput screening of one or more genetic factors. DNA or RNA expression associated with a gene can be investigated through PNA hybridization, which can then be used to estimate the relation between gene and a disease.

In another example, an array can be used to identify one or more biomarkers. Biomarkers can be used for the diagnosis, prognosis, treatment, and management of diseases. Biomarkers may be expressed, or absent, or at a different level in an individual, depending on the disease condition, stage of the disease, and response to disease treatment. Biomarkers can be, e.g., DNA, RNA, PNA, proteins (e.g., enzymes such as kinases), sugars, salts, fats, lipids, or ions.

Arrays can also be used for therapeutic purposes, e.g., identifying one or more bioactive agents. A method for identifying a bioactive agent can comprise applying a plurality of test compounds to an array and identifying at least one test compound as a bioactive agent. The test compounds can be small molecules, aptamers, oligonucleotides, chemicals, natural extracts, peptides, proteins, fragments of antibodies, antibody like molecules, or antibodies. In some embodiments, test compounds are hybridizing DNA, RNA or PNA sequences. The bioactive agent can be a therapeutic agent or modifier of therapeutic targets. Therapeutic targets can include phosphatases, proteases, ligases, signal transduction molecules, transcription factors, protein transporters, protein sorters, cell surface receptors, secreted factors, and cytoskeleton proteins.

In one aspect, also provided are arrays for use in medical diagnostics. An array can be used to determine a response to administration of drugs or vaccines. For example, an individual's response to a vaccine can be determined by detecting the gene expression levels of the individual by using an array with PNA chains or PNA-DNA chimeric oligonucleotide chains representing particular genes associated with the induced immune response. Another diagnostic use is to test an individual for the presence of biomarkers, wherein samples are taken from a subject and the sample is tested for the presence of one or more biomarkers.

Arrays can also be used to stratify patient populations based upon the presence or absence of a biomarker that indicates the likelihood a subject will respond to a therapeutic treatment. The arrays can be used to identify known biomarkers to determine the appropriate treatment group. For example, a sample from a subject with a condition can be applied to an array. Binding to the array may indicate the presence of a biomarker for a condition. Previous studies may indicate that the biomarker is associated with a positive outcome following a treatment, whereas absence of the biomarker is associated with a negative or neutral outcome following a treatment. Because the patient has the biomarker, a health care professional may stratify the patient into a group that receives the treatment.

In some embodiments, a method of detecting the presence or absence of a expressed gene of interest in a sample can include obtaining an array disclosed herein and contacted with a sample suspected of comprising the DNA or RNA sequence of a gene of interest; and determining whether the gene of interest is expressed in the sample by detecting the presence or absence of binding to one or more features of the array. In some embodiments, the DNA or RNA sequence of the gene of interest can be obtained from a bodily fluid, such as amniotic fluid, aqueous humour, vitreous humour, bile, blood serum, breast milk, cerebrospinal fluid, cerumen, chyle, endolymph, perilymph, feces, female ejaculate, gastric acid, gastric juice, lymph, mucus, peritoneal fluid, pleural fluid, pus, saliva, sebum, semen, sweat, synovial fluid, tears, vaginal secretion, vomit, or urine.

In some embodiments, a method of identifying a vaccine candidate can include obtaining an array disclosed herein contacted with a sample derived from a subject previously administered the vaccine candidate, wherein the sample comprises a plurality of DNA or RNA sequences; and determining the binding specificity of the plurality of DNA or RNA sequences to one or more features of the array. In some embodiments, the features comprise a plurality of distinct, nested, overlapping PNA chains comprising subsequences derived from a known nucleotide sequence.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1: Synthesis of a PNA Sequence

A location-specific PNA sequence synthesis is performed on an array as follows: A wafer is spin-coated at 2000-4000 rpm (preferably 2500-3000 rpm) for 10-180 seconds (preferably for 60-120 seconds) with a photoresist composition comprising a photobase generator as described above. The wafer and photoresist is exposed to 248 nm ultraviolet light in a deep ultra violet scanner tool according to a pattern defined by a photomask, wherein the locations exposed to ultraviolet light undergo base generation due to the presence of a photobase generator in the photoactive coupling solution in the photoresist. The expose energy can be from 1 mJ/cm² to 100 mJ/cm² (preferably 30-60 mJ/cm²).

After UV exposure, the surface of the wafer is post baked in a bake module. The post bake temperature can vary between 75° C. to 115° C., for a duration of at least 60 seconds (but not usually exceeding 180 seconds). The base generated at the UV-exposed regions removes a protection group on the amino groups. The photoresist is then stripped.

The free amino group is then coupled to activated R2-acetic acid by spin coating R2-acetic acid with activation agents and reacted in a bake module with the temperature varying from 55° C. to 115° C. for 60-240 seconds. Excess R2-acetic acid is removed.

Following addition of activated R2-acetic acid, a displacement reaction with a mono-amino protected ethylenediamine is performed by addition of a displacement mixture onto the surface of the wafer. This displacement mixture is spin coated on the wafer and reacted in a bake module with the temperature varying from 55° C. to 115° C. for 30 seconds-300 seconds (preferably 120 seconds). In some embodiments, a mono-amino protection group can be any amino protection group as mentioned earlier.

Next, a coupling reaction is performed with the peptide nucleic acid monomer acetic acid. The displaced amine is coupled to the activated PNA monomer acetic acid by spin coating the activated PNA monomer acetic acid with activation agents and allowing to react for 90-300 seconds.

Optionally, a capping solution coat is applied on the surface to prevent any non-reacted amino groups on the substrate from reacting to the next coupling molecule. The capping solution includes a solvent, a polymer, and a coupling molecule. The capping solution is spin-coated on the wafer at 1500-3500 rpm for at least 30 seconds and reacted in a bake module with the temperature varying from 55° C. to 95° C. for 30 seconds-90 seconds (preferably 60 seconds) to complete one cycle.

This entire cycle can be repeated as desired with different nucleic acid monomers each time to obtain desired PNA sequences at specific sites on an array.

### Example 2: Yield of long PNA Sequence

A 64-mer length PNA sequence AGCAATTTTGCATCTTTTAAAAGGGGTTTTCCCCAATATTATGGTTCGTCCCCCTTTAAAGGAT (SEQ ID NO: 1) is grown on the wafer as described in Example 1 and the yield of the sequence polymerization is calculated using fluorescein process as described: To determine the yield of the sequence, 2% by weight of Fluorescein carboxylic acid 5(6)-FAM (Anaspec) activated with 2 equivalents of diisopropylcarbodiimide (VWR). 2 equivalents of Hydroxybenzotriazole (Hobt) (Anapsec) was spin coated on the wafer at 3000 rpm for 60 seconds and baked in a bake module at 65° C. for 5 minutes. The wafer was then washed with Ethylenediamine (VWR) for 10 minutes, then washed with N-methyl-pyrrolidone (NMP) for 2 minutes, then washed with IPA for 40 seconds and spun-dry. Fluorescence was then measured on a confocal microscope to determine the yield of the sequence at each addition.

This example shows the step yield data for the above 64-mer PNA sequence. To measure step yield via fluorescence, a fluorescent dye molecule was coupled to the PNA sequence in order to determine the coupling efficiency. The amount of fluorescein dye coupled gives a direct measure of the amount of sequence grown.

The formula used to calculate step yield was: Step yield=(Fₙ/F₁)^{1/n-1}, where F₁ and Fₙ denotes the fluorescein coupling intensity read out from a fluorescent scanner device at the first step and the nth step. The coupling yield was calculated using the formula E=10^{1/C·log (F)} where F equals fraction of full length and C=number of couplings=length−1. The fraction of full length F is then given by F=10^{(N+1)·log (E/100%)}.

TABLE 1
Step yield of PNA sequence

| Length | Fluorescein Intensity |
|---|---|
| 1 | 65520.14 |
| 2 | 65146.6752 |
| 3 | 64716.70715 |
| 4 | 64088.95509 |
| 5 | 63954.36828 |
| 6 | 63397.96528 |
| 7 | 63328.22751 |
| 8 | 63157.2413 |
| 9 | 62689.87771 |
| 10 | 62470.46314 |
| 11 | 62276.80471 |
| 12 | 62027.69749 |
| 13 | 61760.97839 |
| 14 | 61155.7208 |
| 15 | 60690.93732 |
| 16 | 60508.86451 |
| 17 | 60194.21842 |
| 18 | 59821.01426 |
| 19 | 59623.60491 |
| 20 | 59033.33123 |
| 21 | 58584.67791 |
| 22 | 58479.22549 |
| 23 | 58315.48366 |
| 24 | 57889.78063 |
| 25 | 57542.44194 |
| 26 | 57237.467 |
| 27 | 56859.69972 |
| 28 | 56615.20301 |
| 29 | 56162.28138 |
| 30 | 55623.12348 |
| 31 | 55361.6948 |
| 32 | 54940.94592 |
| 33 | 54682.72348 |
| 34 | 54628.04075 |
| 35 | 54098.14876 |
| 36 | 53979.13283 |
| 37 | 53682.2476 |
| 38 | 53617.8289 |
| 39 | 53317.56906 |
| 40 | 52821.71567 |
| 41 | 52568.17143 |
| 42 | 52321.10103 |
| 43 | 52090.88818 |
| 44 | 51882.52463 |
| 45 | 51633.48851 |
| 46 | 51525.05819 |
| 47 | 51447.7706 |
| 48 | 50979.59589 |
| 49 | 50525.87748 |
| 50 | 50429.87832 |
| 51 | 50303.80362 |
| 52 | 50193.13525 |
| 53 | 50097.7683 |
| 54 | 49972.52387 |
| 55 | 49482.79314 |
| 56 | 49096.82735 |
| 57 | 48851.34322 |
| 58 | 48445.87707 |
| 59 | 48189.11392 |
| 60 | 47899.97924 |
| 61 | 47646.10935 |
| 62 | 47469.81874 |
| 63 | 47251.45758 |
| 64 | 46996.29971 |

Table 1 shows the fluorescence signal intensity at each monomer layer. Table 2 shows the step yield efficiency for the coupling steps, the total yield for the 64-mer, 75-mer, and 100-mer. The total yield for the 75-mer and 100-mer were calculated based on the individual step yield. The coupling efficiency of each PNA monomer was calculated to be greater than 99.4% in each instance across the entire length of each of the 64-mer PNA chain.

TABLE 2
Results of PNA sequencing on array

| | Yield |
|---|---|
| Step yield of each coupled monomer | 99.4% |
| Total yield of 64-mer length sequence | 71.73% |
| Theoretical yield of 75-mer length sequence | 67.74% |
| Theoretical yield of 100-mer length sequence | 59.49% |

Next, we determined the distribution of sequence lengths on the array based on the synthesis of a PNA sequence of a defined length (e.g., a 64 mer). At each coupling step, the length of a sequence where a desired coupling does not occur becomes fixed at that length when a capping solution is used. The distribution of lengths according to the step yield for each sequence length less than the full sequence is given by the following equation:

F(N)=10^{(N+1)·log (E/100%)}−10^{(N)·log (E/100%)}

where F(N) is the proportion of sequences on the array at a length N that are less than the full length sequence, and where E is the average coupling efficiency percentage. The precise value of E at each length N can also be used to generate an exact number of oligomers at each length.

The proportion of full length sequence is given by the following equation:

F(N)=10^{(N)·log (E/100%)}

where F(N) is the proportion of sequences on the array of a full length sequence (no further coupling steps), and where E is the average coupling efficiency.

### Example 3: Hybridization of PNA Sequence to Oligonucleotide DNA Sequence

To determine the biological activity of a PNA sequence, 64-mer length PNA sequences GTGGAAATTTGACATAGTCTCAGATGCCTATTaTATTACCGAGAGAGTAGTCTGAAATGCCTTA (SEQ ID NO: 2) (match sequence), GTGGAAATTTGACATAGTCTCAGATGCCTATTcTATTACCGAGAGAGTAGTCTGAAATGCCTTA (SEQ ID NO: 3) (single base mismatch sequence), and GTGGAAATTTTGTTTTGAAGGACTGTATATAAtTGTGTCATAGTTTGTTACCTAAAATGCCTTA (SEQ ID NO: 4) (mismatch sequence) were synthesized on an array according to the method described above.

Oligonucleotide DNA sequence complimentary to the match sequence TAAGGCATTTCAGACTACTCTCTCGGTAATAtAATAGGCATCTGAGACTATGTCAAATTTCCAC (SEQ ID NO: 5) (complimentary pair sequence) was synthesized (IDT) and labelled with Atto-633 dye (Atto-tec) by techniques well known to one skilled in the art.

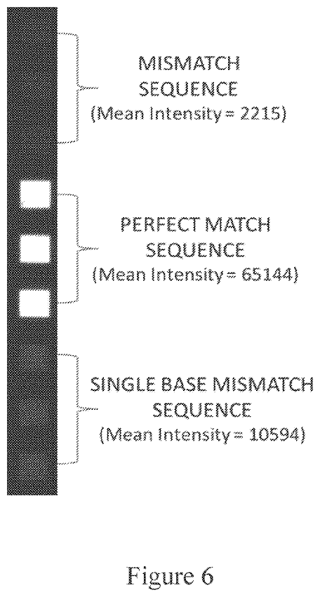

Hybridization of the chip, which contains the match sequence, the single base mismatch sequence and the mismatch sequence, was performed by labelling the complimentary pair sequence, which was diluted 1:1000 in 0.1× Ssarc Buffer (60 mM sodium chloride (Sigma), 6 mM sodium citrate (Sigma), 0.72 weight % N-lauroylsarcosine sodium salt solution (Sigma)). Hybridization was performed in a hybridization chamber at 45° C. for 2 hours. The chips were then washed in 0.1× Ssarc buffer 40° Celsius for 5 minutes twice followed by rinsing in DI Water. The chip was then scanned on a confocal microscope. Results showing identification of a single nucleobase variation using the above method are provided in FIG. 6. The results validate the sensitivity and specificity of identifying a specific DNA sequence (including discrimination among single nucleotide sequence variants) using hybridization to an microarray comprising the specific complementary PNA sequence.

### Example 4: Synthesis of a PNA-DNA Chimera

### Part 1—Synthesis of PNA Sequence

To generate a PNA-DNA chimera on the surface of an array, a PNA oligo of a desired sequence at a specific site was first synthesized on a chip according to the protocol provided in Example 1.

### Part 2 —Synthesis of PNA-DNA Chimera Step

Addition of a sequence-specific DNA nucleotide to the end of a PNA sequence at specific locations on an array is performed as follows: The wafer comprising PNA sequences is spin-coated at 2000-4000 rpm (preferable 2500-3000 rpm) for 10-180 seconds preferably for 60-120 seconds with the photoresist and is exposed to 248 nm ultraviolet light in a deep ultra violet scanner tool according to a pattern defined by a photomask, wherein the locations exposed to ultraviolet light undergo base generation due to the presence of a photobase generator in the photoactive coupling solution in the photoresist. The expose energy can be from 1 mJ/cm² to 100 mJ/cm² (preferably 30-60 mJ/cm²).

After UV exposure, the surface of the wafer is post baked in a bake module. The post bake temperature can vary between 75° C. to 115° C., for a duration of at least 60 seconds (but not usually exceeding 180 seconds). The generated base deprotects the protection of amino groups of the PNA sequences in the exposed regions. The photoresist is then stripped.

Reverse DNA amidites are activated with phosphoramidite activation solution (including e.g., a tetrazole catalyst) and then coupled to the free amine.

Optionally, a capping solution coat is applied on the surface to prevent any non-reacted amino groups on the substrate from reacting to the next coupling molecule. The capping solution includes a solvent, a polymer, and a coupling molecule. The capping solution is spin-coated on the wafer at 1500-3500 rpm for at least 30 seconds and reacted in a bake module with the temperature varying from 55° C. to 95° C. for 30 seconds-90 seconds (preferably 60 seconds) to complete one cycle. The phosphite-triester formed in the coupling step is then converted to a stable form which is achieved by iodine oxidation in the presence of water and pyridine.

### Part 3—Reverse DNA Oligonucleotide Synthesis

A location-specific DNA sequence reverse synthesis (5′ to 3′) is performed at the end of each PNA-DNA chimera on the array as follows: A wafer is spin-coated at 2000-4000 rpm (preferably 2500-3000 rpm) for 10-180 seconds (preferably for 60-120 seconds) with a photoresist composition comprising a photobase generator as described above. The wafer and photoresist is exposed to 248 nm ultraviolet light in a deep ultra violet scanner tool according to a pattern defined by a photomask, wherein the locations exposed to ultraviolet light undergo acid generation due to the presence of a photoacid generator in the photoactive coupling solution. The expose energy can be from 1 mJ/cm² to 100 mJ/cm² (preferably 30-60 mJ/cm²).

After UV exposure, the surface of the wafer is post baked in a bake module. The post bake temperature can vary between 75° C. to 115° C., for a duration of at least 60 seconds (but not usually exceeding 180 seconds). The acid generated deprotects the protection of hydroxyl groups of the 3′-end of DNA sequence in the exposed regions. The photoresist is then stripped.

Reverse DNA amidites are activated with phosphoramidite activation solution (including e.g., a tetrazole catalyst) and then coupled to the free hydroxyl group.

Optionally, a capping solution coat is applied on the surface to prevent any non-reacted amino groups on the substrate from reacting to the next coupling molecule. The capping solution includes a solvent, a polymer, and a coupling molecule. The capping solution is spin-coated on the wafer at 1500-3500 rpm for at least 30 seconds and reacted in a bake module with the temperature varying from 55° C. to 95° C. for 30 seconds-90 seconds (preferably 60 seconds) to complete one cycle. The phosphite-triester formed in the coupling step is then converted to a stable form which is achieved by iodine oxidation in the presence of water and pyridine.

### Example 5: Yield of PNA Sequence & PNA-DNA Chimera

A 31-mer length PNA sequence along with 3 DNA oligomers AGCAATTTTGCATCTTTTAAAAGGGGTTTTC-(CGC) (SEQ ID NO: 6) with (CGC) being the DNA oligomer portion of the sequence was grown on a wafer as described in Example 3 and step yield of the sequence at each addition was determined using as the fluorescein-based step yield detection process as described: 2% by weight of Fluorescein carboxylic acid 5(6)-FAM (Anaspec) activated with 2 equivalents of diisopropylcarbodiimide (VWR) and 2 equivalents of Hydroxybenzotriazole (Hobt) (Anapsec) was spin coated on the wafer at 3000 rpm for 60 seconds and baked in a bake module at 65° C. for 5 minutes. To calculate the yield of the DNA oligomer, 6-FAM phosphoramidite (Jena Biosciences) was activated with 2 equivalents of Benzylthio-tetrazole (TCI) in acetonitrile and spin coated on the wafer at 3000 rpm for 60 seconds and reacted at room temperature for 5 minutes. The wafer was then washed with Ethylenediamine (VWR) for 10 minutes, then washed with N-methyl-pyrrolidone (NMP) for 2 minutes, and then washed with IPA for 40 seconds and spun-dry. Fluorescence was then measured on a Nikon AIR confocal microscope to determine the yield of the sequence. Results are provided in Table 3. From the results in Table 3, the following average step yields for each type of addition were obtained:
Step yield of each layer of PNA sequence=99.4%
Step yield of PNA-DNA chimera step=97.4%
Step yield of DNA oligomer=98.96%

TABLE 3
Step yield of PNA-DNA Chimera Sequence

| Length | Fluorescein Intensity |
|---|---|
| 1 | 65520.14 |
| 2 | 65146.6752 |
| 3 | 64716.70715 |
| 4 | 64088.95509 |
| 5 | 63954.36828 |
| 6 | 63397.96528 |
| 7 | 63328.22751 |
| 8 | 63157.2413 |
| 9 | 62689.87771 |
| 10 | 62470.46314 |
| 11 | 62276.80471 |
| 12 | 62027.69749 |
| 13 | 61760.97839 |
| 14 | 61155.7208 |
| 15 | 60690.93732 |
| 16 | 60508.86451 |
| 17 | 60194.21842 |
| 18 | 59821.01426 |
| 19 | 59623.60491 |
| 20 | 59033.33123 |
| 21 | 58584.67791 |
| 22 | 58479.22549 |
| 23 | 58315.48366 |
| 24 | 57889.78063 |
| 25 | 57542.44194 |
| 26 | 57237.467 |
| 27 | 56859.69972 |
| 28 | 56615.20301 |
| 29 | 56162.28138 |
| 30 | 55623.12348 |
| 31 | 55361.6948 |
| 32 | 53940.94592 |
| 33 | 53382.72348 |
| 34 | 52828.04075 |

### Example 6: Hybridization of PNA-DNA Chimera to Oligonucleotide DNA Sequence and Extension with Polymerase

To determine the biological activity of a PNA-DNA chimera sequence synthesized per the above protocol, a 34-mer length PNA-DNA chimera sequence 5′-GTGGAAATTTGACATAGTCTCAGATGCCTAT(TAT)-3′ (SEQ ID NO: 7) was synthesized according to Example 3 with (TAT) being the DNA oligomer portion of the PNA-DNA chimera. Four oligonucleotide DNA sequences complimentary to the sequence of the PNA-DNA chimera with one additional nucleotide were synthesized (IDT).

S1:
(SEQ ID NO: 8)
CACCTTTAAACTGTATCAGAGTCTACGGATAATAa
S2:
(SEQ ID NO: 9)
CACCTTTAAACTGTATCAGAGTCTACGGATAATAc
S3:
(SEQ ID NO: 10)
CACCTTTAAACTGTATCAGAGTCTACGGATAATAg
S4:
(SEQ ID NO: 11)
CACCTTTAAACTGTATCAGAGTCTACGGATAATAt

A primer extension reaction to detect incorporation of the correct complementary nucleotide in a primer extension reaction was performed. Alexa 405 ddATP, Alexa 488 ddCTP, Alexa 555 ddGTP, Alexa 647 ddTTP were synthesized by techniques well known to one skilled in the art. Hybridization and polymerase extension on each of the 4 chips were performed as follows. Oligomers S1, S2, S3 and S4 were diluted 1:1000 (100 nM) in 1× DNA Polymerase Buffer (Clontech), 20 nmol of MgCl2, 1 unit Titanium Taq DNA Polymerase (Clontech), and all 4 labelled ddNTPs labelled monomers (each 25 pmoles). Hybridization was done in a hybridization chamber at 55° C. for 30 minutes followed by washing the chips in 0.1× Ssarc buffer at 40° C. for 5 minutes twice followed by rinsing in DI Water. The chip was then scanned on a Nikon A1R confocal microscope which included the 4 wavelengths of the dyes used in the ddNTPs and results are depicted in Table 4.

TABLE 4
Hybridization and Extension of PNA-DNA Chimera Sequence

| Sequence | 405 nm | 488 nm | 561 nm | 640 nm |
|---|---|---|---|---|
| S1 (a) | 950.02 | 875.54 | 1047.8 | 65124.23 |
| S2 (c) | 1051.3 | 954.2 | 65531.25 | 875.6 |
| S3 (g) | 800.3 | 65014.78 | 802.5 | 1068.9 |
| S4 (t) | 65121.13 | 1012.98 | 946.6 | 780.9 |

### Example 7: Genotyping Using PNA Sequence Hybridization

Testing of the PNA synthesis for a genotyping SNP-based application was performed. Genotyping of MTHFR region, with the well-known mutations, C677T and A1298C, were tested using 20 DNA samples. The DNA samples had a known genotyping result which were determined using Real-Time PCR.

PNA Sequences were as follows:

(SEQ ID NO: 12)
GGAGAAGGTGTCTGCGGGAG(C)CGATTTCATCATCACGCAGC,
(SEQ ID NO: 13)
GGAGAAGGTGTCTGCGGGAG(T)CGATTTCATCATCACGCAGC
(SEQ2),
(SEQ ID NO: 14)
GGAGGAGCTGACCAGTGAAG(A)AAGTGTCTTTGAAGTCTTCG
(SEQ3),
(SEQ ID NO: 15)
GGAGGAGCTGACCAGTGAAG(C)AAGTGTCTTTGAAGTCTTCG
(SEQ4).

PNA sequences were synthesized on a chip using the methods given above. The location of the SNP is indicated in ( ) region (surrounded by parentheses) and is synthesized in the middle of the sequence.

DNA were extracted from the samples (buccal swabs) using methods known to one skilled in the art. A standard PCR reaction using forward primer and biotin-labelled reverse primers was performed on the extracted DNA samples. Hybridization of the PCR product on the chip was performed with the PCR product (20 ul) diluted in hybridization buffer 0.1× Ssarc Buffer 60 mM sodium chloride (Sigma) (80 ul), 6 mM sodium citrate (Sigma), 0.72 weight % N-lauroylsarcosine sodium salt solution (Sigma). Hybridization was done in a hybridization chamber at 55° C. for 2 hours followed by washing the chips in 0.1× Ssarc buffer 40° C. for 5 minutes twice. This was followed by an incubation with 1 ng/ml Atto 488 Streptavidin (Rockland) diluted in PBS buffer, washing the chips in PBS Buffer twice and rinsing in DI Water. The chip was then scanned on a Nikon A1R confocal microscope and results are depicted in Table 5 and Table 6.

TABLE 5
677C > T Mutation Results (PNA)

| Sample | Original | SEQ ID | SEQ ID | Ratio | Calculated |
|---|---|---|---|---|---|
| ID | Result | NO: 12 (C) | NO: 13 (T) | (C/T) | Result |
| MT1 | Homozygous | 65521.21 | 18343.16 | 3.571969606 | Homozygous |
| | Wild C/C | | | | Wild C/C |
| MT2 | Homozygous | 65227.42 | 17390.41 | 3.750769533 | Homozygous |
| | Wild C/C | | | | Wild C/C |
| MT3 | Homozygous | 65093.45 | 18262.83 | 3.564258661 | Homozygous |
| | Wild C/C | | | | Wild C/C |
| MT4 | Homozygous | 65386.72 | 16498.11 | 3.963285491 | Homozygous |
| | Wild C/C | | | | Wild C/C |
| MT5 | Homozygous | 65245.82 | 17957.35 | 3.633376862 | Homozygous |
| | Wild C/C | | | | Wild C/C |
| MT6 | Homozygous | 65399.12 | 15087.35 | 4.334698937 | Homozygous |
| | Wild C/C | | | | Wild C/C |
| MT7 | Homozygous | 65408.94 | 16166.17 | 4.046038115 | Homozygous |
| | Wild C/C | | | | Wild C/C |
| MT8 | Heterozygous | 32656.73 | 29003.56 | 1.125955917 | Heterozygous |
| | C/T | | | | C/T |
| MT9 | Heterozygous | 31140.4 | 28860.93 | 1.078981169 | Heterozygous |
| | C/T | | | | C/T |
| MT10 | Heterozygous | 30317.09 | 27075.3 | 1.119732376 | Heterozygous |
| | C/T | | | | C/T |
| MT11 | Heterozygous | 29953.05 | 30101.53 | 0.99506736 | Heterozygous |
| | C/T | | | | C/T |
| MT12 | Heterozygous | 34884.62 | 32957.31 | 1.058478984 | Heterozygous |
| | C/T | | | | C/T |
| MT13 | Heterozygous | 28468.43 | 30134.87 | 0.944700608 | Heterozygous |
| | C/T | | | | C/T |
| MT14 | Heterozygous | 29909.26 | 26689.12 | 1.12065366 | Heterozygous |
| | C/T | | | | C/T |
| MT15 | Homozygous | 16202.94 | 65103.75 | 0.248878751 | Homozygous |
| | Mutant T/T | | | | Mutant T/T |
| MT16 | Homozygous | 16759.95 | 65465.38 | 0.256012415 | Homozygous |
| | Mutant T/T | | | | Mutant T/T |
| MT17 | Homozygous | 18019.34 | 65327.52 | 0.275830768 | Homozygous |
| | Mutant T/T | | | | Mutant T/T |
| MT18 | Homozygous | 15153.13 | 65116.35 | 0.232708529 | Homozygous |
| | Mutant T/T | | | | Mutant T/T |
| MT19 | Homozygous | 16837.25 | 65198.8 | 0.258244784 | Homozygous |
| | Mutant T/T | | | | Mutant T/T |
| MT20 | Homozygous | 16315.66 | 65431.64 | 0.249354288 | Homozygous |
| | Mutant T/T | | | | Mutant T/T |
| No | No Template | 931.04 | 1010.87 | | No Template |
| Template | Control | | | | Control |

TABLE 6
1298A > C Mutation Results (PNA)

| Sample | Original | SEQ ID | SEQ ID | Ratio | Calculated |
|---|---|---|---|---|---|
| ID | Result | NO: 14 (A) | NO: 15 (C) | (A/C) | Result |
| MT1 | Homozygous | 65379.46 | 18466.16 | 3.540501111 | Homozygous |
| | Wild A/A | | | | Wild C/C |
| MT2 | Homozygous | 65471.94 | 15279.46 | 4.284964259 | Homozygous |
| | Wild A/A | | | | Wild C/C |
| MT3 | Homozygous | 65031.65 | 16581.28 | 3.92199215 | Homozygous |
| | Wild A/A | | | | Wild C/C |
| MT4 | Homozygous | 65219.55 | 17032.97 | 3.829018075 | Homozygous |
| | Wild A/A | | | | Wild C/C |
| MT5 | Homozygous | 65122.87 | 15781.57 | 4.126514029 | Homozygous |
| | Wild A/A | | | | Wild C/C |
| MT6 | Homozygous | 65211.42 | 16471.62 | 3.959016782 | Homozygous |
| | Wild A/A | | | | Wild C/C |
| MT7 | Homozygous | 65284.01 | 16098.4 | 4.055310466 | Homozygous |
| | Wild A/A | | | | Wild C/C |
| MT8 | Heterozygous | 25864.99 | 26219.09 | 0.986494573 | Heterozygous |
| | A/C | | | | C/T |
| MT9 | Heterozygous | 27535.6 | 33971.8 | 0.810542862 | Heterozygous |
| | A/C | | | | C/T |
| MT10 | Heterozygous | 29798.31 | 25928.75 | 1.149238201 | Heterozygous |
| | A/C | | | | C/T |
| MT11 | Heterozygous | 31306.14 | 30412.04 | 1.02939954 | Heterozygous |
| | A/C | | | | C/T |
| MT12 | Heterozygous | 34735.37 | 26137.17 | 1.328964459 | Heterozygous |
| | A/C | | | | C/T |
| MT13 | Heterozygous | 34293.84 | 32393 | 1.058680579 | Heterozygous |
| | A/C | | | | C/T |
| MT14 | Heterozygous | 34029.48 | 29563.14 | 1.151077998 | Heterozygous |
| | A/C | | | | C/T |
| MT15 | Homozygous | 17863.25 | 65072.67 | 0.274512326 | Homozygous |
| | Mutant C/C | | | | Mutant T/T |
| MT16 | Homozygous | 15361.46 | 65400.42 | 0.234883201 | Homozygous |
| | Mutant C/C | | | | Mutant T/T |
| MT17 | Homozygous | 16092.89 | 65285.28 | 0.246501049 | Homozygous |
| | Mutant C/C | | | | Mutant T/T |
| MT18 | Homozygous | 16038.93 | 65184.73 | 0.246053485 | Homozygous |
| | Mutant C/C | | | | Mutant T/T |
| MT19 | Homozygous | 16752.2 | 65398.01 | 0.256157641 | Homozygous |
| | Mutant C/C | | | | Mutant T/T |
| MT20 | Homozygous | 16298.91 | 65253.44 | 0.249778556 | Homozygous |
| | Mutant C/C | | | | Mutant T/T |
| No | No Template | 931.04 | 1010.87 | | No Template |
| Template | Control | | | | Control |

In this method, the SNP location is ideally close to the center of the sequence synthesized.

### Example 8: Genotyping Using PNA-DNA Chimera and Primer Extension

Testing of the PNA-DNA chimera for a genotyping SNP-based application was performed. Genotyping of MTHFR region, with the well-known mutations, C677T and A1298C, were tested using 20 DNA samples. The DNA samples had a known genotyping result which were determined using Real-Time PCR. 34-mer length PNA-DNA chimera primer sequences 5′-CTGAAGCACTTGAAGGAGAAGGTGTCTGCGG(GAG)-3′ (SEQ ID NO: 16) for the 677C>T mutation and 5′- CTGAAGATGTGGGGGGAGGAGCTGACCAGTG(AAG)-3′ (SEQ ID NO: 17) for the 1298A>C mutation were synthesized according to the methods given above (with DNA nucleotide portion of the PNA-DNA chimera enclosed in parentheses).

DNA were extracted from the samples (buccal swabs) using methods known to one skilled in the art. A standard PCR reaction using forward and reverse primers was performed on the extracted DNA samples. Hybridization and polymerase extension on each of chips were performed as follows. The PCR product was mixed in 1× DNA Polymerase Buffer (Clontech), 20 nmol of MgCl₂, 1 unit Titanium Taq DNA Polymerase (Clontech), and all 4 labelled ddNTPs labelled monomers (each at 25 pmol). Hybridization was done in a hybridization chamber at 55° C. for 30 minutes followed by washing the chips in 0.1× Ssarc buffer 40° C. for 5 minutes twice followed by rinsing in DI Water. The chip was then scanned on a Nikon A1R confocal microscope which included the 4 wavelengths of the dyes used in the ddNTPs and results are depicted in Table 7 and Table 8.

TABLE 7
677C > T Mutation Results (PNA-DNA Chimera)

| Sample | Original | 405 nm | 488 nm | 561 nm | 640 nm | Ratio | Calculated |
|---|---|---|---|---|---|---|---|
| ID | Result | (A) | (C) | (G) | (T) | (C/T) | Result |
| MT1 | Homozygous | 987.88 | 65381.38 | 1068.03 | 1022.91 | 63.9170406 | Homozygous |
| | Wild C/C | | | | | | Wild C/C |
| MT2 | Homozygous | 1079.22 | 65379.69 | 1003.71 | 1088.7 | 60.0529898 | Homozygous |
| | Wild C/C | | | | | | Wild C/C |
| MT3 | Homozygous | 890.15 | 65426.33 | 1054.07 | 984.99 | 66.4233444 | Homozygous |
| | Wild C/C | | | | | | Wild C/C |
| MT4 | Homozygous | 1019.28 | 65082.83 | 1072.37 | 1005.67 | 64.7158909 | Homozygous |
| | Wild C/C | | | | | | Wild C/C |
| MT5 | Homozygous | 1054.92 | 65520.82 | 885.87 | 919.46 | 71.26010919 | Homozygous |
| | Wild C/C | | | | | | Wild C/C |
| MT6 | Homozygous | 856.58 | 65086.76 | 1063 | 1081.46 | 60.18415845 | Homozygous |
| | Wild C/C | | | | | | Wild C/C |
| MT7 | Homozygous | 1014.84 | 65048.37 | 1012.61 | 1020.77 | 63.72480578 | Homozygous |
| | Wild C/C | | | | | | Wild C/C |
| MT8 | Heterozygous | 1086.58 | 28940.21 | 916.77 | 25640.7 | 1.128682524 | Heterozygous |
| | C/T | | | | | | C/T |
| MT9 | Heterozygous | 1045.85 | 34602.61 | 903.71 | 33874.1 | 1.021506402 | Heterozygous |
| | C/T | | | | | | C/T |
| MT10 | Heterozygous | 920.14 | 30597.21 | 1085.44 | 32891.41 | 0.930249266 | Heterozygous |
| | C/T | | | | | | C/T |
| MT11 | Heterozygous | 1065.78 | 26105.81 | 883.95 | 25875.75 | 1.00889095 | Heterozygous |
| | C/T | | | | | | C/T |
| MT12 | Heterozygous | 1087.05 | 28716.64 | 1028.78 | 30765.41 | 0.933406706 | Heterozygous |
| | C/T | | | | | | C/T |
| MT13 | Heterozygous | 1096.23 | 30296.19 | 1097.06 | 29260.09 | 1.035410007 | Heterozygous |
| | C/T | | | | | | C/T |
| MT14 | Heterozygous | 999.32 | 31152.26 | 1083.28 | 28772.12 | 1.082723831 | Heterozygous |
| | C/T | | | | | | C/T |
| MT15 | Homozygous | 959.46 | 920.49 | 983.83 | 65264.85 | 0.014103917 | Homozygous |
| | Mutant T/T | | | | | | Mutant T/T |
| MT16 | Homozygous | 999.03 | 1017.52 | 1098.91 | 65228.95 | 0.015599209 | Homozygous |
| | Mutant T/T | | | | | | Mutant T/T |
| MT17 | Homozygous | 908.97 | 1031.03 | 1083.42 | 65170.19 | 0.015820577 | Homozygous |
| | Mutant T/T | | | | | | Mutant T/T |
| MT18 | Homozygous | 1053.12 | 1000.45 | 961.54 | 65066.09 | 0.015375905 | Homozygous |
| | Mutant T/T | | | | | | Mutant T/T |
| MT19 | Homozygous | 1070.32 | 1075.46 | 1009.19 | 65326.99 | 0.016462721 | Homozygous |
| | Mutant T/T | | | | | | Mutant T/T |
| MT20 | Homozygous | 1059.73 | 915.48 | 854.63 | 65287.66 | 0.014022252 | Homozygous |
| | Mutant T/T | | | | | | Mutant T/T |
| No | No | 1005.16 | 931.04 | 1018.61 | 1010.87 | | No |
| Template | Template | | | | | | Template |
| | Control | | | | | | Control |

TABLE 8
1298 A > C Mutation Results (PNA-DNA Chimera)

| Sample | Original | 405 nm | 488 nm | 561 nm | 640 nm | Ratio | Calculated |
|---|---|---|---|---|---|---|---|
| ID | Result | (A) | (C) | (G) | (T) | (A/C) | Result |
| MT1 | Homozygous | 65488.6 | 860.42 | 898.56 | 1053.57 | 76.11236373 | Homozygous |
| | Wild A/A | | | | | | Wild A/A |
| MT2 | Homozygous | 65344.12 | 942.87 | 1005.5 | 859.47 | 69.30342465 | Homozygous |
| | Wild A/A | | | | | | Wild A/A |
| MT3 | Homozygous | 65439.64 | 943 | 1040.81 | 1048.08 | 69.39516437 | Homozygous |
| | Wild A/A | | | | | | Wild A/A |
| MT4 | Homozygous | 65352.13 | 1015.9 | 850.32 | 978.43 | 64.32929422 | Homozygous |
| | Wild A/A | | | | | | Wild A/A |
| MT5 | Homozygous | 65258.17 | 1047.97 | 999.32 | 858.43 | 62.27102875 | Homozygous |
| | Wild A/A | | | | | | Wild A/A |
| MT6 | Homozygous | 65327.7 | 871.43 | 984.33 | 971.42 | 74.96609022 | Homozygous |
| | Wild A/A | | | | | | Wild A/A |
| MT7 | Homozygous | 65222.79 | 1032.19 | 1093.43 | 894.49 | 63.18874432 | Homozygous |
| | Wild A/A | | | | | | Wild A/A |
| MT8 | Heterozygous | 32437.6 | 29322.89 | 851.72 | 897.02 | 1.106221113 | Heterozygous |
| | A/C | | | | | | A/C |
| MT9 | Heterozygous | 28901.78 | 32234.2 | 972.78 | 930.13 | 0.896618498 | Heterozygous |
| | A/C | | | | | | A/C |
| MT10 | Heterozygous | 31930.24 | 34009.27 | 902.33 | 1072.67 | 0.938868726 | Heterozygous |
| | A/C | | | | | | A/C |
| MT11 | Heterozygous | 31187.77 | 33959.45 | 1066.13 | 858.62 | 0.918382659 | Heterozygous |
| | A/C | | | | | | A/C |
| MT12 | Heterozygous | 35160.88 | 34129.73 | 1085.9 | 978.16 | 1.030212662 | Heterozygous |
| | A/C | | | | | | A/C |
| MT13 | Heterozygous | 26032.4 | 34987.94 | 980.73 | 1047.35 | 0.744039232 | Heterozygous |
| | A/C | | | | | | A/C |
| MT14 | Heterozygous | 32902.79 | 33518.34 | 1024.53 | 998.06 | 0.981635427 | Heterozygous |
| | A/C | | | | | | A/C |
| MT15 | Homozygous | 853.23 | 65405.7 | 866.06 | 1096.1 | 0.013045193 | Homozygous |
| | Mutant C/C | | | | | | Mutant C/C |
| MT16 | Homozygous | 861.56 | 65469.41 | 1045.55 | 1093.83 | 0.013159734 | Homozygous |
| | Mutant C/C | | | | | | Mutant C/C |
| MT17 | Homozygous | 948.8 | 65335.08 | 1094.53 | 1025.03 | 0.014522061 | Homozygous |
| | Mutant C/C | | | | | | Mutant C/C |
| MT18 | Homozygous | 853.16 | 65190.22 | 964.72 | 901.27 | 0.013087239 | Homozygous |
| | Mutant C/C | | | | | | Mutant C/C |
| MT19 | Homozygous | 1014.49 | 65003.94 | 1015.66 | 922.79 | 0.015606592 | Homozygous |
| | Mutant C/C | | | | | | Mutant C/C |
| MT20 | Homozygous | 926.32 | 65437.85 | 945.85 | 1063.8 | 0.014155722 | Homozygous |
| | Mutant C/C | | | | | | Mutant C/C |
| No | No | 1043.98 | 1043.85 | 986.73 | 1073.34 | | No |
| Template | Template | | | | | | Template |
| | Control | | | | | | Control |

In this method, the sequence synthesized on the chip contains the region just before the SNP location, thereby enabling the polymerase to selectively add the matched oligomer corresponding to the SNP identity.

The PNA-DNA chimera was able to hybridize to the DNA sequence and extend accurately according to the corresponding match DNA monomer. The PNA-DNA chimera is able to obtain a high Match/Mismatch Ratio which would accurately identify SNP-based genotyping results. Ratio for Match/Mismatch sequence is 3.5-4 for PNA sequence while it is 65-70 or PNA-DNA chimera sequence. Thus, PNA-DNA chimera with high yield of the sequence and the ability to perform a polymerase extension step on chip due to the DNA oligomer present provides a high-throughput, high accuracy system for various genomics applications including, but not limited to, SNP-based genotyping and DNA sequencing.

1-39. (canceled)

40. A composition comprising a PNA-DNA chimera array, said array comprising features attached to a surface at positionally-defined locations, each of said features comprising a plurality of PNA-DNA chimera polymers having a 5′ end and a 3′ end;
each PNA-DNA chimera polymer comprising a PNA chain and a DNA chain each comprising a 5′ end and a 3′ end, said 5′ end of the PNA chain is coupled to the surface, and the 3′ end of the PNA chain is coupled to the 5′ end of the DNA chain.

41. The composition of claim 40, wherein the length of the PNA chain is at least 30 bases.

42. The composition of claim 40, wherein the length of the PNA chain is about 3 bases.

43. The composition of claim 40, wherein the purity of each feature with regards to the fraction of full-length predetermined PNA-DNA chimeric polymer is a fraction F of the full-length predetermined PNA-DNA chimeric polymer of each feature having a predetermined sequence and a predetermined full-length sequence length N being characterized by F=10(N+1)·log(E/100%) with an average coupling efficiency E of at least 98.5% for coupling each PNA monomer or DNA monomer of the predetermined sequence.

44. A method of making a PNA-DNA chimera array, said array comprising features attached to a surface at positionally-defined locations, each of said features comprising a plurality of PNA-DNA chimera polymers; the method comprising:
(a) providing a PNA array comprising features attached to a surface at positionally-defined locations, each of said features comprising a plurality of PNA polymers, wherein the amine group of each PNA polymer is protected by a protecting group;
(b) generating a pattern with a photomask on the PNA array;
(c) exposing a photoresist to UV light through said photomask and generating a base from a photobase generator in said pattern on said array as a result of said exposure to said UV light, wherein said base cleaves the protecting group from the amine group;
(d) coupling a first DNA monomer to said unprotected amine group.

45. The method of claim 44, wherein the DNA monomer comprises a protected reverse phosphoamidite.

46. The method of claim 45, further comprising, activating the protected reverse phosphoamidite and coupling the activated reverse phosphoamidite in the DNA monomer and the unprotected amino group in the PNA polymer.

47. The method of claim 45, wherein the protected reverse phosphoamidite comprises a dimethoxytrityl (“DMT”) or a 5′-O-(α-methyl-6-nitropiperonyloxycarbonyl) (MeNPOC) protection group.

48. The method of claim 46, wherein the activating comprises removing the protection group.

49. The method of claim 44, wherein said photobase generator is selected from the group consisting of: 1,3-Bis[(2-nitrobenzyl)oxycarbonyl-4-piperidyl]propane, 1,3-Bis[1-(9-fluorenylmethoxycarbonyl)-4-piperidyl]propane, 1,5,7-triazabicyclo[4.4.0]dec-5-enyl-phenylglyoxylate, 1,5,7-triazabicyclo[4.4.0]dec-5-enyl-4-nitrophenylglyoxylate, 1,5,7-triazabicyclo[4.4.0]dec-5-enyl-tetraphenylborate, 1,8-Diazabicyclo[5.4.0]undec-7-enyl-tetraphenylborate, 1-Phenacyl-(1-azonia-4-azabicyclo[2,2,2]octane)-tetraphenylborate, and 1-Naphthoylmethyl-(1- azonia-4- azabicyclo[2,2,2]octane)-tetraphenylborate.

50. The method of claim 49, wherein said photobase generator is 1,3-Bis[(2-nitrobenzyl)oxycarbonyl-4-piperidyl]propane.

51. The method of claim 44, further comprising (e) coupling a second DNA monomer to the first DNA monomer.

52. The method of claim 51, wherein the method step (e) is repeated.

53. The method of claim 51, wherein the coupling step in (e) comprises a reverse DNA oligonucleotide synthesis.

54. The method of claim 44, wherein said coupling of the DNA monomer is performed on a plurality of sites on said array simultaneously.

55. The method of claim 44, wherein said plurality of PNA-DNA chimera polymers comprises a distribution of lengths characterized by a coupling efficiency of at least 90%, 91%, 92%, 93%, 94%, 95%, 96%, 97%, 98%, 98.5%, 99%, or 99.5%.

56. The method of claim 44, wherein the length of the PNA polymer chain is a least 30 PNA monomers.

57. The method of claim 44, wherein each PNA-DNA chimeric polymer chain is from 5 to 100 monomers in length.

58. A method of analyzing a sample, said sample comprising nucleic acids obtained from a subject, comprising:
contacting said sample with an array produced by the method of claim 40 under conditions that promote hybridization between said sample and said array;
detecting a signal from said array, wherein said signal indicates the presence, absence or amount of sample hybridized to said array at one or more of said feature locations; and
analyzing said signal, thereby analyzing said sample.

59. The method of claim 58, wherein the analyzing comprises determining the presence or absence of a SNP present in said sample based on said signal.

60. The method of claim 59, wherein said method further comprises carrying out a primer extension reaction following said hybridization between said sample and said array.
